(19) United States
(12) Patent Application Publication
(10) Pub. No.: US 2019/0328721 A1
(43) Pub. Date: Oct. 31, 2019

(54) METHOD OF ACTIVATING DENDRITIC CELLS

(71) Applicant: KOREA INSTITUTE OF SCIENCE AND TECHNOLOGY, Seongbuk-gu (KR)

(72) Inventors: GiHoon NAM, Seongbuk-gu (KR); Yoo Soo Yang, Seongbuk-gu (KR); In-San Kim, Seongbuk-gu (KR); Seung-Yoon Park, Seongbuk-gu (KR)

(73) Assignee: KOREA INSTITUTE OF SCIENCE AND TECHNOLOGY, Seongbuk-gu (KR)

(21) Appl. No.: 16/394,223

(22) Filed: Apr. 25, 2019

Related U.S. Application Data
(60) Provisional application No. 62/662,340, filed on Apr. 25, 2018.

(51) Int. Cl. A61K 31/4409; A61P 35/00; A61P 37/04; A61K 39/395

(52) U.S. Cl. CPC A61K 31/4409; A61P 35/00; A61K 2039/505; A61K 39/39541; A61P 37/04

(57) ABSTRACT

Described are methods for activating dendritic cells using a Rho-related protein kinase (ROCK) inhibitor, optionally in combination with one or more of an immunogenic cell death-inducing chemotherapeutic, photodynamic therapy, or radiation therapy. The methods and treated cells are useful, for example, in treating cancer, optionally in combination with one or more of an immunogenic cell death-inducing chemotherapeutic, immune checkpoint inhibitor, photodynamic therapy, or radiation therapy.

### CROSS-REFERENCES TO RELATED APPLICATIONS

This application claims priority under 35 USC § 119(e) to U.S. provisional application Ser. No. 62/662,340 filed Apr. 25, 2018, the contents of which are incorporated in their entirety by reference herein.

### TECHNICAL FIELD

The present invention is drawn to a method for activating cells. In particular, the present invention is drawn to a method for activating dendritic cells.

### BACKGROUND

Dendritic cells are immune cells contained in the mammalian immune system. Their main function is to treat pathogens and present them on the surface for other cells in the immune system. In other words, dendritic cells function as antigen-presenting cells. In addition, dendritic cells function as mediators between innate and acquired immune responses. Immature dendritic cells constantly sample foreign antigens from the environment in order to detect pathogens such as viruses and bacteria. This is accomplished by a pattern recognition receptor (PRR) such as toll like receptors (TLRs). TLRs recognize distinctive chemical moieties that appear in some groups of pathogens, and once they come into contact with pathogens, they become activated mature dendritic cells, and begin to migrate to the lymph nodes. Immature dendritic cells digest pathogens via phagocytosis, break down proteins, and then display their fragments on the cell surface using major histocompatibility complex (MHC). At the same time, they increase the ability to activate T cells by increasing the amount of cell surface receptors such as CD80, CD86 and CD40, which are used as co-receptors in T cell activation. They also induce the migration of dendritic cells into the spleen through the blood vessel or into the lymph node through the lymphatic system by increasing the expression of CCR7. Dendritic cells are therein used as antigen presenting cells to present the antigen of pathogens to helper T cells, cytotoxic T cells (killer T cells), and B cells or activate the cells via non-antigen specific co-stimulatory signals.

In addition, according to recent studies it has been shown that cancer-associated dendritic cells, such as CD103-positive dendritic cells, play an important role in the T cell-cancer immune response by transporting cancer antigens to the draining lymph node and cross-presenting the cancer antigen to cytotoxic T cells.

On the other hand, Rho-associated protein kinase (ROCK) is a kinase belonging to the AGC (PKA/PKG/PKC) family of serine/threonine kinases. ROCK is generally divided into ROCK1 and ROCK2, and human ROCK1 and ROCK2 are major downstream effectors of GTPase RhoA. Rho GTPase is a small G-protein that plays an important role in signal transduction that regulates cell migration, growth and differentiation.

ROCK inhibitors are inhibitors of the function of ROCK. They function as inhibitors of one or more of apoptosis, regeneration of neurites, inhibition of agonist-induced Ca²⁺ sensitization in myosin phosphorylation and smooth muscle contraction. ROCK inhibitors have been reported to be effective and safe therapeutics in clinical trials in a variety of cardiovascular disorders such as angina, coronary vasospasm, hypertension, pulmonary hypertension, heart failure, and cerebral ischemia.

The ROCK inhibitor fasudil has been used for cerebral angiography in Japan since 1995. In the United States, clinical studies on fasudil for Raynaud's phenomenon treatment, atherosclerosis and hypercholesterolemia are underway. In addition, recently, a pharmaceutical composition for prevention and treatment of dry eye syndrome using the ROCK inhibitor Y27632 has been patented (Korean Patent Laid-Open Publication No. 10-2015-025566).

However, the relationship between ROCK and dendritic cells is not yet known.

### SUMMARY

In an aspect of the present invention, provided is a method for activating dendritic cells in a subject comprising administering a Rho-related protein kinase (ROCK) inhibitor to the subject.

In another aspect of the present invention, provided is a method for activating dendritic cells in a subject, comprising:

administering a ROCK inhibitor and an immunogenic cell death-inducing chemotherapeutics to the subject; or

administering a ROCK inhibitor to the subject and applying photodynamic therapy or radiation therapy to the subject.

In another aspect of the present invention, provided is a method of selectively activating cancer-associated dendritic cells in a cell population comprising dendritic cells or progenitor cells of dendritic cells, comprising treating the cell population with a ROCK inhibitor.

In another aspect of the present invention, provided is a method for treating a cancer patient, comprising:

administering a cell population comprising dendritic cells or progenitor cells of dendritic cells treated with a ROCK inhibitor and an immunogenic cell death-inducing chemotherapeutics to the cancer patient; or

administering a cell population comprising dendritic cells or progenitor cells of dendritic treated with a ROCK inhibitor and applying a photodynamic therapy or radiation therapy to the cancer patient.

In another aspect of the present invention, provided is a method for treating a cancer patient, comprising: administering a therapeutically effective amount of a ROCK inhibitor and an immune checkpoint inhibitor to the cancer patient.

### EFFECTS OF THE INVENTION

According to one aspect, a composition for enhancing an immune response and method using the same as described herein improves an anti-cancer immune response by an innate immune response, an adaptive immune response, or a combination thereof, and can be effectively used for cancer immunotherapy.

### BRIEF DESCRIPTION OF DRAWINGS

FIG. 1 is a schematic diagram showing a mechanism of enhanced phagocytosis of cancer cells by dendritic cells leading to activated CD8 T cells due to combination therapy with a ROCK inhibitor and doxorubicin (a kind of immunogenicity apoptosis-inducing anticancer agent), according to an embodiment of the present invention.

FIG. 2A is a pair of histograms showing the results of flow cytometry analysis of bone marrow cells (BM cells, left) and bone marrow-derived macrophages (BMDM, right) differentiated therefrom using anti-F4/80 antibodies.

FIG. 2B is a pair of histograms showing the results of flow cytometry of bone marrow cells (BM cells, left) and bone marrow derived dendritic cells (BMDCs, right) differentiated therefrom using anti-CD11c antibodies.

FIG. 2C is a series of 2-D histograms showing the result of flow cytometry analysis of phagocytosis of cancer cells by bone marrow-derived macrophages (BMDMs) when BMDMs stained with CellTracker Deep Red and pretreated with a vehicle (control) or Y27632 (a ROCK inhibitor) and a colon cancer cell line (CT26.CL25) or a malignant melanoma cell line (B16F10-Ova) stained with CellTracker CMFDA were co-cultured.

FIG. 2D is a graph showing the phagocytosis ratio of cancer cells by bone marrow-derived macrophages pretreated with a vehicle (control) or Y27632.

FIG. 2E is a series of 2-D histograms showing the result of flow cytometry analysis of phagocytosis of cancer cells by a bone marrow-derived dendritic cell (BMDCs), when BMDCs stained with CellTracker Deep Red and pretreated with a vehicle (control) or Y276321 and a colon cancer cell line (CT26.CL25) or a malignant melanoma cell line (B16F10-Ova) stained with CellTracker CMFDA were co-cultured.

FIG. 2F is a graph showing the phagocytosis ratio of cancer cells by bone marrow-derived dendritic cells pretreated with a vehicle (control) or Y27632.

FIG. 2G is a series of photographs taken with a fluorescence microscope showing phagocytized cancer cells (EC) when the cancer cells co-cultured with bone marrow-derived macrophages (BMDMs, left) pretreated with a vehicle (control) or Y27632 or bone marrow-derived dendritic cells (BMDCs, right) pretreated with a vehicle (control) or Y27632.

FIG. 2H is a graph showing the change in the phagocytosis ratio of cancer cells by BMDMs (left) pretreated with a vehicle (control) or Y27632 or by BMDCs (right) pretreated with a vehicle (control) or Y7632.

FIG. 3 is a series of graphs showing the change in the phagocytosis ratio of cancer cells by BMDMs (left) and BMDCs (right) over time (0→4 h) when Y27632 was used to pretreat BMDMs and BMDCs for 1 hour, respectively, and then after removing Y27632, the BMDMs or BMDCs were treated with a colon cancer cell line (CT26.CL25, left) or a malignant melanoma cell line (B16F10-Ova, right).

FIG. 4 is a series of graphs showing the change in the phagocytosis ratio of cancer cells by BMDMs and BMDCs when Y27632 was used to pretreat only phagocytes (BMDMs and BMDCs) or only on cancer cells (CT26.CL25 and B 16F10-Ova), or both, and then the phagocytes and cancer cells were co-cultured, to confirm whether Y27632 promotes phagocytosis by acting directly on cancer cells.

FIG. 5A is a pair of graphs showing the phagocytosis ratio of a colon cancer cell line (CD26.CL25, left) and malignant melanoma cell line (B16F10-Ova, right) treated with BMDMs pretreated with a vehicle (control) or blebbistatin.

FIG. 5B is a pair of graphs showing phagocytosis ratio of a colon cancer cell line (CT26.CL25) and a malignant melanoma cell line (B16F10-Ova) by BMDCs treated with blebbistatin (an agent capable of inhibiting the movement of the myosin light chain downstream of the ROCK signal pathway).

FIG. 5C is a schematic diagram showing the Rho signaling pathway.

FIG. 6A is a series of histograms showing the results of flow cytometry analyzing apoptosis of a malignant melanoma cell line (B16F10-Ova) using FITC-anti-Annexin V antibodies, when the cell line was untreated, or treated with TRAIL, doxorubicin (0.25 μM and 2.5 μM), vehicle only (control) or Y27632 (30 μM) for 24 hours, respectively.

FIG. 6B is a graph showing the proportion of Annexin V-positive cells in each experimental group as a result of flow cytometry in FIG. 6A.

FIG. 6C is a series of histograms showing the results of flow cytometry analyzing apoptosis of a colon cancer cell line (CT26.CL25) using FITC-anti-Annexin V antibodies, when the cell line was untreated, or treated with TRAIL, doxorubicin (2.5 μM and 25 μM), vehicle only (control) or Y27632 (30 μM) for 24 hours, respectively.

FIG. 6D is a graph showing the proportion of Annexin V-positive cells in each experimental group as a result of the flow cytometry of FIG. 6C.

FIG. 7A shows an administration schedule in experimental animals to examine changes in the number of phagocytic cells among injected CellTracker CFSE-stained apoptotic thymocytes after administration of Y27632 according to an embodiment of the present invention.

FIG. 7B is a series of histograms showing the results of flow cytometry analysis of phagocytic activity on injected apoptotic thymocytes by sorting macrophages (F4/80³⁰ ) and dendritic cells (CD11c⁺) derived from the spleen of experimental animals injected with apoptotic thymocytes according to the administration schedule shown in FIG. 7A and then 1 hour after administration of vehicle or Y27632.

FIG. 7C is a graph showing the results of measurement of phagocytosis ratio of injected apoptotic thymocytes of macrophages (MΦs) and dendritic cells (DCs) as a result of flow cytometry in FIG. 7B.

FIG. 8A shows an administration schedules in tumor model animals to confirm the effect of Y27632 on the tumor model animals prepared by injecting a colon cancer cell line (CT26.CL25, left) or a malignant melanoma cell line (B16F10-Ova, right), respectively.

FIG. 8B is a pair of graphs showing the results of measurement of tumor weights depending on whether Y27632 was administered or not to tumor model animals prepared by injecting a colon cancer cell line (CT26.CL25, left) or a malignant melanoma cell line (B16F10-Ova, right), respectively.

FIG. 8C is a pair of graphs showing the change in the body weights over time after administration of vehicle or Y27632, respectively in tumor model animals produced by injecting a colon cancer cell line (CT26.CL25, left) or a malignant melanoma cell line (B16F10-Ova, right), respectively.

FIG. 8D is a series of graphs showing the results of measurement of the tumor volumes over time after administration of vehicle or Y27632, respectively, in tumor model animals prepared by injecting a colon cancer cell line (CT26.CL25). The left graph shows statistical results, the upper right graph shows changes in tumor volumes of individual animals in the control group and the lower right graph shows changes in tumor volume of individual animals in the Y27632-treated group.

FIG. 8E is a series of graphs showing the results of measurement of the tumor volumes over time after administration of vehicle or Y27632, respectively, in tumor model animals prepared by injecting a malignant melanoma cell line (B16F10-Ova). The left graph shows statistical results, the upper right graph shows changes in tumor volume of individual animals in the control group and the lower right graph shows changes in tumor volume of individual animals in the Y27632-treated group.

FIG. 8F is a graph showing the result of measuring the tumor volumes over time after administering a vehicle, 10 mg/kg of Y27632 or 20 mg/kg of fasudil to tumor model animals prepared by injecting CT26 cells.

FIG. 9A shows an administration schedule for analyzing the effect of administration of Y27632 on tumor model animals prepared by subcutaneously injecting a CT26.CL25 cell line into immunodeficient nude mice.

FIG. 9B is a series of graphs showing the results of measurement of the tumor volumes over time after administration of a vehicle or Y27632, respectively, to tumor model animals prepared by subcutaneously injecting a CT26.CL25 cell line into immunodeficient nude mice.

FIG. 10A shows an administration schedule in an experimental animal model to investigate the effect of phagocytic cell-deficiency on the effect of Y27632 in a tumor model animal prepared by subcutaneously injecting a colon cancer cell line (CT26.CL25).

FIG. 10B shows an administration schedule in an experimental animal model to confirm the role of T cells in the effect of Y27632 on a tumor model animal prepared by subcutaneous injection of a colon cancer cell line (CT26.CL25).

FIG. 10C is a graph showing the results of measuring the relative phagocytic activity of macrophages (F4/80⁺ MΦ) and dendritic cells (CD11c⁺ DC) when Y27632 and clodronate liposome were administered according to the administration schedule shown in FIG. 10A compared with a control (PBS liposome).

FIG. 10D is a graph showing the results of measuring the relative activity cell immunity of CD8 T cells) and CD4 T cells when Y27632 and antagonizing antibodies were administered according to the administration schedule shown in FIG. 10b compared with a control (nonspecific antibodies).

FIG. 10E is a series of graphs showing the results of measurement of changes in the tumor volumes over time after administration of Y27632 and/or clodronate liposome to experimental animals according to the schedule shown in FIG. 10A.

FIG. 10F is a graph showing the results of measurement of changes in tumor volumes over time after administration of Y27632 and/or antagonizing antibodies (anti-CD4 antibodies or anti-CD8 antibodies) to experimental animals according to the administration schedule shown in FIG. 10B.

FIG. 11A is a series of histograms showing the results of flow cytometry analysis to analyze phagocytic activities of macrophages (MΦ) and dendritic cells (DCs) against cancer cells expressing mCherry, which were derived from excised tumor tissues from tumor model animals prepared by injecting CT26.CL25 colon cancer cells expressing mCherry.

FIG. 11B is a pair of graphs quantifying the phagocytosis ratio of cancer cells from the flow cytometry analysis results shown in FIG. 11A.

FIG. 11C is a graph showing the results of analysis of the expression level of INF-γ in the tumor-draining lymph nodes depending on whether clodronate was administered or not to an experimental animal according to the administration schedule shown in FIG. 10A.

FIG. 11D is a graph showing the results of analysis of the expression level of INF-γ by CD8 T cells in the tumor-draining lymph nodes using an anti-CD8a magnetic bead in an experimental animal administered according to the administration schedule shown in FIG. 10B.

FIG. 12A shows an administration schedule in experimental animals for the analysis of the anti-cancer immune memory activity of Y27632 according to one embodiment of the present invention.

FIG. 12B is a graph showing tumor-free survival rates over time after administration of a vehicle (control) or Y27632 to tumor model animals prepared by injecting a CT26.CL25 colon cancer cell line, removing the tumor tissue after a certain time and re-injecting the same colon cancer cell line to another site according to the administration schedule shown in FIG. 12A.

FIG. 13A is a pair of graphs showing the proportion of CD40-positive dendritic cells (left) and CD86-positive dendritic cells (right), which are dendritic cell maturation markers, in the tumor drain lymph node isolated from B16F10-Ova bearing tumor models animals administered a vehicle or Y27632 according to the same administration schedule as in Example 2-1 analyzed by a flow cytometer.

FIG. 13B shows the results of pretreatment of Y27632 (30 μM) for 1 hour on bone marrow-derived dendritic cells (BMDCs) stained with CellTracker CMFDA, followed by co-cultivation of CT26.CL25 for 4 hours, and then, floating cancer cells were removed by removing the supernatant. The histograms (top) show the degree of expression of the maturation markers CD40 and CD86 by flow cytometry analysis of each ratio of CD40-positive and CD86-positive dendritic cells, and the left lower graphs represent the results of the flow cytometry analysis of the proportion of CD40- and CD86-BMDCs, and the right lower graphs represent relative median fluorescence intensity (relative MFI) showing the results of measuring the proportion of dendritic cells.

FIG. 14A shows an administration schedule in experimental animal models to investigate the effect of the ROCK inhibitor on T cell priming. Y27632 was administered in the B16F10-Ova-bearing cancer model according to the administration schedule described in Example 2-1, followed by injecting OT-1 T cells, staining with CFSE (carboxyfluorescein succinimidyl ester), and examining the degree of T cell proliferation after 3 days.

FIG. 14B is a pair of histograms showing the results of flow cytometry analysis of the number of proliferation of OT-1 T cells after injecting Y27632 and CFSE-stained OT-1 T cells according to the administration schedule shown in FIG. 14a. As the intensity of fluorescence decreases, the number of OT-1 T cell proliferation increases.

FIG. 14C is a graph comparing the number of propagation times of the control group and the Y27632-administered group by grouping them from the result of FIG. 14B.

FIG. 14D is a pair of graphs showing the expression level of IFN-γ in isolated macrophages (MΦ) and dendritic cells (DCs) from the tumor-draining lymph nodes (left) and tumor tissues (right) in B16F10-Ova-bearing tumor model animals administered a vehicle or Y27632 according to the same administration schedule as in Example 2-1 and co-cultured with OT-1 T cells.

FIG. 14E is a pair of graphs showing the results of flow cytometry analysis sorting IFN-γ-positive T cells from OT-1 T cells co-cultured with dendritic cells isolated from the tumor-draining lymph node of the tumor model animals administered a vehicle or Y27632 using anti-IFN-γ antibodies.

FIG. 15A is a graph showing the results of cytometry analysis to analyze the effect of the ROCK inhibitor on CD103 DCs. A vehicle or Y27632 was administered to B16F10-Ova-bearing cancer model animals according to the administration schedule described in Example 2-1, and then tumor-draining lymph nodes were excised to detect various dendritic cell markers (CD11b, CD103, and CD8) using antibodies specific thereto.

FIG. 15B is a graph showing the results of cytometry analysis to analyze the effect of the ROCK inhibitor on CD103 DCs. A vehicle or Y27632 was administered to B16F10-Ova-bearing cancer model animals according to the administration schedule described in Example 2-1, and then tumor tissues were excised to detect various dendritic cell markers (CD11b, and CD103) using antibodies specific thereto.

FIG. 15C is a graph showing the results of flow cytometry analysis for the cross-presentation activity of dendritic cells, which is one of functions capable of activating T cells of DCs. B16F10-Ova-bearing mice were administered a vehicle or Y27632 according to the administration schedule described in Example 2-1, the tumor-draining lymph nodes were excised and then cancer antigen H-2K^{b}-Ova-positive tumor cells were sorted by a flow cytometer using antibodies specific to the cancer antigen H-2K^{b}-Ova, which is displayed on MHC-1.

FIG. 15D is a graph showing the results of flow cytometry analysis for the cross-presentation activity of dendritic cells, which is one of functions capable of activating T cells of DCs. B16F10-Ova-bearing mice were administered a vehicle or Y27632 according to the administration schedule described in Example 2-1, the tumor tissues were excised and then cancer antigen H-2K^{b}-Ova-positive tumor cells were sorted by a flow cytometer using antibodies specific to the cancer antigen H-2K^{b}-Ova, which is displayed on MHC-1.

FIG. 15E is a graph showing the proportion of CD40-positive DCs analyzed by flow cytometry using anti-CD40 antibodies to analyze the effect of Y27632 on the B16F10-Ova tumor model animals. B16F10-Ova-bearing mice were administered a vehicle or Y27632 according to the administration schedule described in Example 2-1, the tumor-draining lymph nodes were excised and then CD40-positive DCs were sorted by a flow cytometer using antibodies specific to CD40, which is a DC maturation marker and one of markers dendritic cells capable of activating T cells.

FIG. 15F is a graph showing the proportion of CD40-positive DCs analyzed by flow cytometry using anti-CD40 antibodies to analyze the effect of Y27632 on the B16F10-Ova tumor model animals. B16F10-Ova-bearing mice were administered a vehicle or Y27632 according to the administration schedule described in Example 2-1, the tumor tissues were excised and then CD40-positive DCs were sorted by a flow cytometer using antibodies specific to CD40, which is a DC maturation marker and one of markers dendritic cells capable of activating T cells.

FIG. 15G is a graph showing the results of phagocytic activity of DCs isolated from tumor tissues against cancer cells after administering Y27632 to B16F10-Ova-bearing tumor model animals according to the administration schedule described in Example 2-1.

FIG. 16A is a series of histograms showing the results of flow cytometry analysis on phagocytic activities of CellTracker Deep Red-stained bone marrow-derived dendritic cells (BMDCs) against cancer cells when they were co-cultured with CellTracker CMFDA-stained colon cancer cells (CT26.CL25, left) or malignant melanoma cells (B16F10-Ova, right) which were pretreated with doxorubicin and a vehicle or Y27632 according to one embodiment of the present invention.

FIG. 16B is a graph showing the phagocytosis ratio of BMDCs against cancer cells from the flow cytometry analysis results of FIG. 16A.

FIG. 16C is a series of fluorescent microscopic images showing phagocytic action of bone marrow-derived dendritic cells (BMDCs) against cancer cells when the BMDCs were co-cultured with pH rodo SE-stained colon cancer cells (CT26.CL25) pretreated with doxorubicin, under the condition of treating with a vehicle or Y27632 using pHrodo SE staining.

FIG. 16D is a graph representing the calculated phagocytosis ratio of BMDCs against cancer cells from the analysis result of FIG. 16C.

FIG. 16E is a series of histograms showing the results of flow cytometry analysis using antibodies specific to CD40 and CD86, respectively, which are dendritic cell maturation markers. Dendritic cells expressing the markers were sorted using the antibodies after CellTracker CMFDA-stained BMDCs pretreated with Y27632 were co-cultured with CT26.CL26 colon cancer cells treated with doxorubicin, and followed by removing floating cancer cells through withdrawal of supernatant.

FIG. 16F is a graph showing the proportions of CD40-positive and CD86-positive dendritic cells, respectively, from the results of FIG. 16E.

FIG. 16G is a series of histograms showing the results of flow cytometry analysis representing cross-presentation activity which is one of functions of dendritic cells capable of activating T cell, using antibodies specific to H-2K^{b}-Ova, which is a cancer antigen displayed on MHC-1. Dendritic cells expressing the cancer antigen were sorted using the antibodies after CellTracker CMFDA-stained BMDCs pretreated with Y27632 were co-cultured with B16F10-Ova cells treated with a vehicle or doxorubicin, and followed by removing floating cancer cells through withdrawal of supernatant.

FIG. 16H is a graph showing the proportion of dendritic cells cross-presenting cancer antigen (H-2Kb-Ova) from the results of FIG. 16G.

FIG. 17A is a series of 2-D histograms showing the results of flow cytometry analysis representing the degree of phagocytosis of bone marrow-derived dendritic cells (BMDCs) against cancer cells. The CellTracker Deep Red-stained BMDCs were pretreated with a ROCK inhibitor Y27632 and co-cultured with CellTracker CMFDA- stained CT26.CL25 colon cancer cells and CellTracker CMFDA-stained B16F10-Ova malignant melanoma cells in which necrosis was induced by high-temperature treatment, respectively.

FIG. 17B is a graph representing the phagocytosis ratio of dendritic cells against each cancer cell from the analysis result of FIG. 17A.

FIG. 17C is a series of 2-D histograms showing the results of flow cytometry analysis representing the degree of phagocytosis of bone marrow-derived dendritic cells (BMDCs) against cancer cells. The CellTracker Deep Red-stained BMDCs were pretreated with a ROCK inhibitor Y27632 and co-cultured with CellTracker CMFDA- stained CT26.CL25 colon cancer cells and CellTracker CMFDA-stained B16F10-Ova malignant melanoma cells in which non-immunogenic apoptosis was induced by treatment of cisplatin, respectively.

FIG. 17D is a graph representing the phagocytosis ratio of dendritic cells against each cancer cell from the analysis result of FIG. 17C.

FIG. 18A shows an administration schedule in experimental animals for investigating whether an immunogenic cell death-inducing chemotherapeutics can be intensified in combination with a ROCK inhibitor to enhance an anti-cancer immune response according to an embodiment of the present invention.

FIG. 18B is a series of graphs showing the results of measuring the tumor volumes over time after administration of a ROCK inhibitor Y27632 and/or doxorubicin, an immunological apoptosis-inducing anticancer agent to the tumor model.

FIG. 18C is a graph showing the result of measuring the weight of tumor finally removed as a result of FIG. 18B.

FIG. 18D is a pair of graphs showing the results of flow cytometry analysis representing the proportions of CD40-positive and CD-86-positive dendritic cells in the tumor-draining lymph node as a result of FIG. 18B.

FIG. 18E is a graph showing the results of flow cytometry analysis representing cross-presentation activity which is one of functions of dendritic cells capable of activating T cell, using antibodies specific to H-2K^{b}-Ova which is a cancer antigen displayed on MHC-1. Dendritic cells isolated from the tumor-draining lymph nodes of B16F10-Ova-bearing tumor models treated with Y27632 and doxorubicin were sorted using the antibodies.

FIG. 18F is a graph showing the results of measuring the amount of IFN-γ after co-culturing dendritic cells isolated from the tumor-draining lymph node of the B16F10-Ova-bearing tumor model with OT-1T cells.

FIG. 18G is a graph showing the results of measuring the proportion of IFN-γ-positive T cells after co-culturing dendritic cells isolated from the tumor-draining lymph node of the B16F10-Ova-bearing tumor model with OT-1T cells.

FIG. 18H is a series of fluorescent microscopic images representing the result of fluorescence immunohistochemistry analysis using anti-CD8 antibodies after sectioning the tumor tissue extracted from an experimental animal in order to confirm whether CD8⁺T cells are infiltrated into the tumor tissue.

FIG. 18I is a graph showing the results of calculating the density of CD8-positive T cells from the results of FIG. 18H.

FIG. 19A is a graph showing the results of animal experiments to determine the dose of cisplatin showing an effect corresponding to doxorubicin of 5 mg/kg for a comparative experiment of cisplatin, which is a non-immunogenic cell death-inducing chemotherapeutics.

FIG. 19B is a graph showing tumor volumes over times after administration of a ROCK inhibitor Y27632 and/or cisplatin which is a non-immunogenic cell death-inducing chemotherapeutics at the concentration (3 mg/kg) determined from the result of FIG. 19A.

FIG. 19C is a graph showing the results of flow cytometry analysis representing the proportions of CD40-positive and CD86-positive dendritic cells in the tumor-draining lymph node, which is derived from the result of FIG. 19B.

FIG. 19D is a graph showing the relative median fluorescence intensity (relative MFI) calculated from the result of FIG. 19C.

FIG. 19E is a graph showing the results of flow cytometry analysis representing cross-presentation activity of dendritic cells which is one of functions of dendritic cells capable of activating T cells using antibodies capable of analyzing cancer antigen (H-2Kb-Ova) displayed on MHC-1 in dendritic cells. Tumor-draining lymph nodes were isolated from B16F10-Ova-bearing cancer model animal administered Y27632 and cisplatin and dendritic cells cross-representing the cancer antigen were sorted by the antibodies specific to the cancer antigen by a flow cytometer.

FIG. 20A shows an administration schedule in experimental animals for analyzing the anticancer effect of the combination of doxorubicin and Y27632 in a spontaneously occurring cancer model.

FIG. 20B is a graph showing tumor volumes over time after administration of doxorubicin and/or Y27632 in the spontaneously occurring cancer model according to the administration schedule shown in FIG. 20A.

FIG. 20C is a graph showing long-term survival rate after administration of doxorubicin and/or Y27632 in the spontaneously occurring cancer model according to the administration schedule shown in FIG. 20A.

FIG. 20D is a series of fluorescence microscopic images representing fluorescence immunohistochemistry analysis using anti-CD8 antibodies in cancer tissues obtained after the end of the animal experiment of FIG. 20B.

FIG. 20E is a graph showing a result of measuring the density of CD8-positive cells by fluorescence immunohistochemistry analysis of FIG. 20D.

FIG. 20F is a graph showing the results of measuring the amount of IFN-y released in isolated spleen cells from the spontaneously occurring cancer model treated with a cancer antigen.

FIG. 21 is a graph showing the results of flow cytometry analysis to investigate the effect of fasudil on the phagocytosis of apoptotic cancer cells. BMDMs were pretreated with fasudil and then co-cultured with mitoxantrone treated B16F10-Ova malignant melanoma cells and the phagocytosis of BMDMs against the mitoxantrone treated B16F10-Ova cells were analyzed by a flow cytometer.

FIG. 22 is a graph showing the result of cell viability analysis of cancer cells treated with various concentrations of a photosensitizer (FIC NP) alone or with photodynamic therapy (PDT) by irradiating LED light.

FIG. 23A is a graph showing expression level of calreticulin (CRT) in CT26 colon cancer cells treated with various concentration of FIC NP alone (Dark) or with photodynamic therapy (PDT) by irradiating LED light.

FIG. 23B is a histogram representing a flow cytometry analysis of CRT-positive CT26 cancer cells treated with various concentrations of FIC NPs along with photodynamic therapy.

FIG. 23C is a graph showing expression level of calreticulin (CRT) in B16F10 melanoma cells treated with various concentrations of FIC NP alone (Dark) or with photodynamic therapy (PDT) by irradiating LED light.

FIG. 24A is a series of graphs representing phagocytosis rate of BMDMs treated with or without ripasudil against cancer cells treated with or without PDT analyzed by fluorescence microscopic analysis (left) and flow cytometry analysis (right).

FIG. 24B is a series of fluorescence microscopic images of BMDMs treated with or without ripasudil and engulfed B16F10 cancer cells treated with or without PDT.

FIG. 24C is a series of graphs representing the phagocytosis rate of BMDCs treated with or without ripasudil against cancer cells treated with or without PDT analyzed by fluorescence microscopic analysis (left) and flow cytometry analysis (right).

FIG. 24D is a series of fluorescence microscopic images of BMDCs treated with or without ripasudil and engulfed B16F10 cancer cells treated with or without PDT.

FIG. 25A is a schedule of treatment with FIC-PDT and administration of ripasudil in tumor model mice subcutaneously injected with 5×10⁵ B16F10 cells.

FIG. 25B is a graph showing tumor volumes over time after treatment with PDT and/or administration of ripasudil to the tumor model mice according to the administration schedule shown in FIG. 25A.

FIG. 25C is a graph showing the result of measuring the weight of tumor removed as a result of FIG. 25B.

FIG. 25D is a graph showing the result of flow cytometry analysis using anti-CD40 antibodies or anti-CD86 antibodies along with anti-CD11c antibodies in the tumor-draining lymph nodes of tumor model mice treated with ripasudil and/or PDT.

FIG. 25E represents a graph showing ratio of CD8⁺ T cells in tumor tissues excised from tumor model mice treated with ripasudil and/or PDT (left) and a series of fluorescence microscopic images showing fluorescence immunohistochemical analysis of the above tumor tissues.

FIG. 25F is a graph showing expression level of INF-γ secreted by co-cultivation of tumor-draining lymph node cells from tumor model mice treated with ripasudil and/or PDT and UV-treated B16F10 tumor cells.

FIG. 26 is a series of fluorescence microscopic images showing the result of immunohistochemical analysis using anti-PD-L1 antibodies of cryo-sections of tumor tissues excised from tumor model mice treated with ripasudil and/or PDT.

FIG. 27A is a schedule of treatment with FIC-PDT and administration of ripasudil and anti-PD-L1 antibodies to tumor model mice subcutaneously injected with 5×10⁵ B16F10 cells.

FIG. 27B is a graph showing tumor volumes over time after treatment with PDT and/or administration of ripasudil and anti-PD-L1 antibodies to the tumor model mice according to the administration schedule shown in FIG. 27A.

FIG. 28A is a graph showing tumor volumes over time after treatment with fasudil and/or mitoxantrone (MTX) to the tumor model mice.

FIG. 28B is a graph showing the result of measuring the weight of tumor removed as a result of FIG. 28A.

FIG. 29A is a graph representing the results of a flow cytometry analysis showing the ratio of CD45.2⁺CD3⁺CD8⁺ cells in the tumor-draining lymph nodes isolated from tumor model mice administered fasudil and/or MTX.

FIG. 29B is a graph showing the expression level of INF-γ in the tumor-draining lymph nodes excised from tumor model mice administered fasudil and/or MTX.

FIG. 29C is a series of fluorescence microscopic images showing the result of immunohistochemical analysis using anti-CD8a antibodies of cryo-sections of tumor tissues excised from tumor model mice treated with fasudil and/or MTX.

FIG. 30 is a series of fluorescence microscopic images showing the result of immunohistochemical analysis using anti-PD-L1 antibodies of cryo-sections of tumor tissues excised from tumor model mice treated with fasudil and/or MTX.

FIG. 31A is a graph showing tumor volumes over time after administration of anti-PD-L1 antibodies, mTX+fasudil combination, or MTX+fasudil+anti-PD-L1 antibodies triple combination, to tumor model mice, respectively.

FIG. 31B is a graph showing the result of measuring the weight of tumor removed as a result of FIG. 31A.

FIG. 31C is a graph showing ratio of CD8⁺ T cells in the tumor tissues excised from tumor model mice administered anti-PD-L1 antibodies, MTX+fasudil combination, or MTX+fasudil+anti-PD-L1 antibodies triple combination to tumor model mice, respectively.

FIG. 31D is a graph showing survival rate of CD8⁺ T cell in the tumor tissues excised from tumor model mice administered anti-PD-L1 antibodies, MTX+fasudil combination, or MTX+fasudil+anti-PD-L1 antibodies triple combination to tumor model mice, respectively.

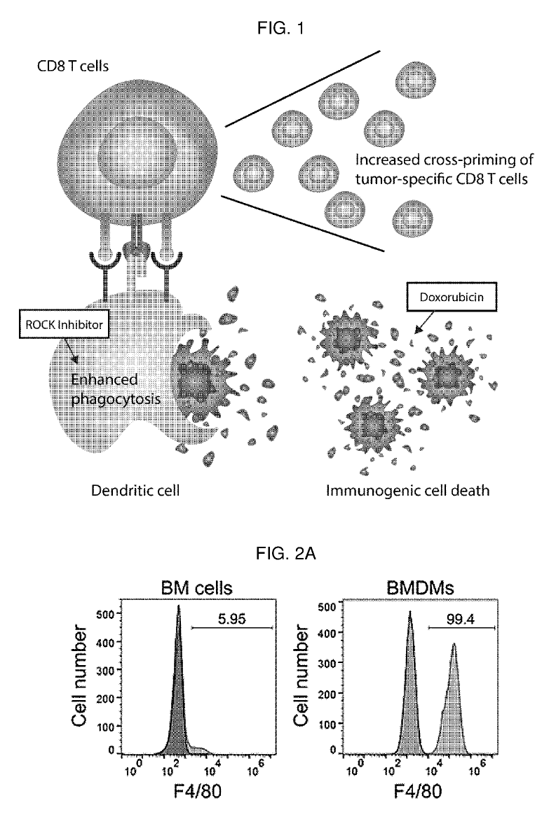

### DETAILED DESCRIPTION

### Definitions

The term “Rho-related protein kinase (ROCK)” as used herein means a phosphorylating enzyme belonging to the AGC (PKA/PKG/PKC) family of serine/threonine kinases, generally divided into ROCK1 and ROCK2, which acts on the skeleton to regulate cell migration.

The term “dendritic cells (DCs)” as used herein means immune cells that constitute the immune system of a mammal. Matured dendritic cells are characterized by having a plurality of twig shaped dendrites, and phagocytize pathogens such as bacteria or viruses and present the processed antigens derived from the pathogens to their surface for other immune cells such as T cells or B cells.

The term “cancer-associated dendritic cells” as used herein refers to dendritic cells that phagocytize cancer cells, translocate to the draining lymph node, and cross-present processed cancer antigens to cytotoxic T cell. They thereby play an important role in T cell-tumor immune response. Representative cancer-associated dendritic cells include CD103-positive dendritic cells in mice and CD141-positive dendritic cells in humans.

The term “immunogenic cell death-inducing chemotherapeutics” as used herein refers to an anticancer agent that kills cancer cells by inducing anticancer immune responses, such as through the activation of dendritic cells caused by treatment of cell proliferation inhibitors or applying radiotherapy or photodynamic therapy, and the specific activation of T cell immune responses thereby, in addition to inducing direct cancer cell death. Immunogenic apoptosis-inducing anticancer drugs are well described in some documents (Kroemer et al. Annu. Rev. Immunol., 31: 51-72, 2013, etc.). The above document is incorporated herein by reference in its entirety.

The term “anthracycline-type anticancer agent” as used herein refers to chemotherapeutic cell cycle-independent anticancer agents derived from Streptomyces peucetius var. caesius. Anthracycline-type anticancer agents are used for the treatment of various cancers including leukemia, lymphoma, breast cancer, stomach cancer, uterine cancer, ovarian cancer, bladder cancer and lung cancer, and are one of most effective anticancer agents among those developed previously. The first anthracycline anticancer drugs discovered was daunorubicin, followed by doxorubicin. In addition, epirubicin, idarubicin, pixantrone, sabarubicin, and valrubicin, etc. have been developed Examples of mechanisms of action of anthracycline-type anticancer agents include inhibiting the proliferation of cancer cells growing rapidly by intercalating between base-pairs of DNA/RNA strands and inhibiting DNA and RNA synthesis thereby; inhibiting transcription and replication in cancer cells by inhibiting the relaxation of DNA strands due to the inhibition of topoisomerase II enzyme activity; inducing damage of DNA, protein and plasma membrane through the formation of iron-mediated free oxygen radicals; and inducing histone expelling from chromatin structure which regulates epigenome and transcripts. Recent studies have shown that doxorubicin increases the Th1 immune response by activating CD4⁺ cells (Park et al., Int. Immunopharmacol. 9(13-14): 1530-1539, 2009), and it was reported that dendritic cells in combination with doxorubicin induced immunogenic cell death of osteosarcoma (Kawano et al., Oncol. Lett. 11: 2169-2175, 2016).

The term “immune checkpoint inhibitor” as used herein refers to a class of drugs that block certain types of immune system cells, such as T lymphocytes, and certain proteins produced by some cancer cells, which prevent T lymphocytes from killing cancer cells. Thus, when these proteins are blocked, the “brake system” of the immune system is released and T lymphocytes can kill cancer cells better. PD-1/PD-L1 and CTLA-4/B7-1/B7-2 are well known as the above-mentioned “immune check point”. Examples of PD-1 inhibitors include pembrolizumab (KEYTRUDA®), nivolumab (OPPDVIO®). Inhibitors of PD-L1, a ligand of PD-1, include atezolizumab (TECCENTRIQ®) and avelumab)(BAVENCIO®). Additionally, ipilimumab (YERVOY®) and the like have been approved by the US FDA as CTLA-4 inhibitors that inhibit the interaction of CTLA-4/B7-1/B7-2. Clinical trials in recent years have shown impressive success in the treatment of patients suffering from some cancers, particularly metastatic melanoma or Hodgkin lymphoma, and there is much potential for clinical trials in other types of cancer patients.

In an aspect of the present invention, provided is a method for activating dendritic cells in a subject comprising administering a Rho-related protein kinase (ROCK) inhibitor to the subject.

According to the method, the ROCK inhibitor may be a ROCK2 selective inhibitor, or a ROCK1/2 pan-inhibitor. The ROCK2 specific inhibitor may be fasudil, KD-025 {2-(3-(4-((1H-indazol-5-yl)amino)quinazolin-2-yl)phenoxy)-N-isopropylacetamide)}, BA-1049, Rho kinase inhibitor V {N-(4-(1H-pyrazol-4-yl)phenyl)-2,3-dihydrobenzo[b][1,4]dioxine-2-carboxamide}, SR3677, or LYC-53976. The ROCK1/2 pan-inhibitor may be ripasudil, RKI-1447, Y-27632, GSK429286A, Y-30141, thiazovivin, GSK180736A, GSK269962A, netrasudil, Y-39983, ZInC00881524, Yf-356{(+)-(R)-4-(1-Aminoethyl)-N-(4-pyridyl) benzamide}, Rho kinase inhibitor IV {(S)-(+)-2-Methyl-4-glycyl-1-(4-methylisoquinolinyl-5-sulfonyl) homopiperazine, H-1152}, Rho kinase inhibitor II {N-(4-Pyridyl)-N′-(2,4,6-trichlorophenyl)urea}, SB772077B, Rho kinase inhibitor III {(3-(4-Pyridyl)-1H-indole)}, K-115, HA1100, rhostatin, CCG-1423 {N-(2-(4-Chloroanilino)-1-methyl-2-oxoethoxy)-3,5-bis(tri-fluoromethyl)benzamide}, cethrin (VX-210), BA-210, BA-1042, BA-1043, BA-1044, BA-1050, BA-1051, BA-1076, BA-215, BA-285, BA-1037, Ki-23095, or AT13148.

According to the method, the dendritic cells may be cancer-associated dendritic cells, and the caner-associated dendritic cells may be CD103- or CD141-positive dendritic cells.

According to the method, the ROCK inhibitor may be administered via a variety of routes of administration including, but not limited to, oral administration, intravenous administration, intramuscular administration, intranasal administration, intraperitoneal administration, subcutaneous administration, intradermal administration, intracardiac administration, intraocular administration, intrathecal administration, intraarticular administration, intraarterial administration, sublingual administration, intravaginal administration, intracranial administration and transmucosal administration.

The term “therapeutically effective amount” as used herein refers to an amount sufficient to significantly increase the immune response when administered to a subject in need of an increased immune response through activation of dendritic cells. The therapeutically effective amount can be appropriately selected depending on the cell or individual being treated and selected by a person skilled in the art. It can be determined according to the severity of the disease, the age, weight, health, sex, sensitivity of the patient to the drug, time of administration, route of administration and rate of excretion, duration of treatment, preparation of used composition, factors including drugs used in combination with or other factors well known in the art. The effective amount may be from about 0.5 μg to about 2 g, from about 1 μg to about 1 g, from about 10 μg to about 500 mg, from about 100 μg to about 100 mg, or from about 1 mg to about 50 mg per composition.

In another aspect of the present invention, provided is a method for activating dendritic cells in a subject, comprising:

administering a ROCK inhibitor and an immunogenic cell death-inducing chemotherapeutics to the subject; or

administering a ROCK inhibitor to the subject and applying photodynamic therapy or radiation therapy to the subject.

According to the method, the ROCK inhibitor may be a ROCK2 selective inhibitor, or ROCK1/2 pan-inhibitor. The ROCK2 specific inhibitor may be fasudil, KD-025 {2-(3-(4-((1H -indazol-5-yl)amino)quinazolin-2-yl)phenoxy)-N-isopropylacetamide)}, BA-1049, Rho kinase inhibitor V {N-(4-(1H-pyrazol-4-yl)phenyl)-2,3-dihydrobenzo[b][1,4]dioxine-2-carboxamide}, SR3677, or LYC-53976. The ROCK1/2 pan-inhibitor may be ripasudil, RKI-1447, Y-27632, GSK429286A, Y-30141, thiazovivin, GSK180736A, GSK269962A, netrasudil, Y-39983, ZInC00881524, Yf-3561{(+)-(R)-4-(1-Aminoethyl)-N-(4-pyridyl) benzamide}, Rho kinase inhibitor IV {(S)-(+)-2-Methyl-4-glycyl-1-(4-methylisoquinolinyl-5-sulfonyl) homopiperazine, H-1152}, Rho kinase inhibitor II {N-(4-Pyridyl)-N′-(2,4,6-trichlorophenyl)urea}, SB772077B, Rho kinase inhibitor III {(3-(4-Pyridyl)-1H-indole)}, K-115, HA1100, rhostatin, CCG-1423 {N-(2-(4-Chloroanilino)-1-methyl-2-oxoethoxy)-3,5-bis(tri-fluoromethyl)benzamide}, cethrin (VX-210), BA-210, BA-1042, BA-1043, BA-1044, BA-1050, BA-1051, BA-1076, BA-215, BA-285, BA-1037, Ki-23095, or AT13148.

According to the method, the immunogenic cell death-inducing chemotherapeutics may be an anthracycline-type anticancer agent, cetuximab, paclitaxel, bleomycin, cyclophosphamide, mitoxantrone or oxaliplatin, and the anthracycline-type anticancer agent may be daunorubicin, doxorubicin, epirubicin, idarubicin, pixantrone, sabarubicin, or valrubicin.

According to the method, the ROCK inhibitor and the immunogenic cell death-inducing chemotherapeutics may be administered simultaneously or sequentially at regular intervals. For example, the dendritic cells in a subject may be activated by administering the ROCK inhibitor, followed by administration of an immunogenic cell death-inducing chemotherapeutic at regular intervals, such as 1 day, 2 days, 3 days, 4 days, or 1 week. The immunogenic cell death-inducing chemotherapeutic can induce direct apoptosis of cancer cells and secondarily activate the dendritic cells, thereby inducing a synergistic effect in the immune response to cancer cells. Alternatively, an immunogenic cell death-inducing chemotherapeutics can be administered first to induce cancer cell death and activation of dendritic cells, and then the ROCK inhibitor can be administered at regular intervals, for example, 1 day, 2 days, 3 days, 4 days, etc., in order to activate the dendritic cells further and to thereby induce a synergistic effect in the immune response against cancer cells.

In another aspect of the present invention, provided is a method of selectively activating cancer-associated dendritic cells in a cell population comprising dendritic cells or progenitor cells of the dendritic cells, comprising treating the cell population with a ROCK inhibitor.

According to the method, the ROCK and the ROCK inhibitor are any as described above.

The methods can be performed in vitro or in vivo. When carried out in vitro, a population of cells comprising dendritic cells can be obtained from the blood or bone marrow of a subject suffering from cancer, such as buffy coat obtained by centrifuging blood or hematopoietic cells isolated from bone marrow, and then treated with the ROCK inhibitor in order to increase the number of cancer-associated dendritic cells and to activate their phagocytic response to cancer cells. Such cell populations comprising cancer-associated dendritic cells activated in vitro may be administered ex vivo to the cancer patient either directly or after proliferation. Optionally, it is possible to amplify the number of cancer-associated dendritic cells and increase the activity thereof in vivo by activating immature dendritic cells or progenitors thereof present in the cancer patient by administering the ROCK inhibitor to the cancer patient.

In another aspect of the present invention, provided is a method for treating a cancer patient, comprising:

administering a cell population comprising dendritic cells or progenitor cells of the dendritic cells treated with a ROCK inhibitor and an immunogenic cell death-inducing chemotherapeutics to the cancer patient; or

administering a cell population comprising dendritic cells or progenitor cells of the dendritic cells treated with a ROCK inhibitor and applying a photodynamic therapy or radiation therapy to the cancer patient.

In the above method, the ROCK inhibitor and the immunogenic cell death-inducing chemotherapeutics may be any as described above.

In this method, the cell population and the immunogenic cell death-inducing chemotherapeutic may be administered simultaneously or sequentially at regular intervals. For example, an immune response to cancer cells can be induced by administering the immunogenic cell death-inducing chemotherapeutics at a predetermined interval, for example, 1 day, 2 days, 3 days, 4 days, or 1 week after the cell population is administered. Alternatively, it is possible to induce an anti-cancer immune response against cancer by first administering the immunogenic cell death-inducing chemotherapeutics to the cancer patient and then administering the cell population at a predetermined interval, for example, 1 day, 2 days, 3 days, 4 days or 1 week.

The immune response may be an innate immune response, an adaptive immune response, or a combination thereof. The innate immune response is also referred to as a nonspecific immune response. The innate immune responses may be due to macrophages, dendritic cells, neutrophils, or a combination thereof. The adaptive immune response is also referred to as an acquired immune response or a specific immune response. Adaptive immune responses are mediated by the presentation of exogenous or endogenous antigens by the action of T cells and B cells; adaptive immune responses include immunological memory.

The immune response may be due to phagocytosis. Phagocytosis is the process by which a cell takes up solid matter, particles, or other cells from the environment. By phagocytosis, the cells can remove pathogens that invade from the outside or cancer cells by intracellular digestion after intake them. The phagocytosis may be the phagocytosis of macrophages, dendritic cells, neutrophils, or a combination thereof. The phagocytic action may be a phagocytic action on cancer cells. When the phagocytosis of cancer cells is increased, adaptive immunity against cancer cells is elevated through the presentation of cancer cells-derived antigens and activation and proliferation of T cells, so that cancer cells are removed via CD8⁺ T cells, and after cancer cells are removed, cancer recurrence can be suppressed through immunological memory.

Photodynamic therapy and radiotherapy are known to induce immunogenic cell death of cancer cells, similar to immunogenic cell death-inducing chemotherapeutics. Thus, instead of or in addition to administering an immunogenic cell apoptosis-inducing anticancer agent in combination with the ROCK inhibitor, it is possible to promote the action of enhancing the cellular immune activity through macrophages and dendritic cells by performing photodynamic therapy or radiotherapy at the same time as or before or after administration of the ROCK inhibitor. Accordingly, in the above methods, the administration of the cell population and photodynamic therapy or radiotherapy may be performed simultaneously or sequentially at regular intervals.

The cancer may be a solid cancer or a non-solid cancer. The solid tumor is a cancerous tumor of the organs such as liver, lungs, breast, and skin. The non-solid cancer is a cancer that develops in the blood, also called a blood cancer. The cancer may be carcinoma, sarcoma, cancer derived from hematopoietic cells, germ cell tumor, or blastoma. The cancer may be selected from the group consisting of, for example, breast cancer, skin cancer, head and neck cancer, pancreatic cancer, lung cancer, colon cancer, colorectal cancer, stomach cancer, ovarian cancer, prostate cancer, bladder cancer, urethral cancer, liver cancer, kidney cancer, papillary carcinoma, melanoma, brain spinal cancer, brain cancer, thymoma, mesothelioma, esophageal cancer, biliary tract cancer, testicular cancer, germ cell tumor, thyroid cancer, parathyroid cancer, cervical cancer, endometrial cancer, lymphoma, myelodysplastic syndromes (MDS), myelofibrosis, acute leukemia, chronic leukemia, multiple myeloma, Hodgkin's disease, endocrine cancer, and sarcoma. The cancer cells may be cells derived from the cancer. The cancer cells may be cells derived from the cancer.

In the methods described herein, the ROCK inhibitor, the immunogenic cell death-inducing chemotherapeutics, and/or the cell population may be formulated in the form of a conventional pharmaceutical composition. The pharmaceutical composition may comprise a pharmaceutically acceptable carrier. The carrier may be an excipient, diluent and/or adjuvant. Examples of suitable carriers include lactose, dextrose, sucrose, sorbitol, mannitol, xylitol, erythritol, maltitol, starch, acacia gum, alginate, gelatin, calcium phosphate, calcium silicate, cellulose, methylcellulose, polyvinyl pyrrolidone, water, physiological saline, buffer such as PBS, methylhydroxybenzoate, propylhydroxybenzoate, talc, magnesium stearate and mineral oil. The composition may include a filler, an anti-coagulant, a lubricant, a wetting agent, a flavoring agent, an emulsifier, a preservative, and the like.

The composition can be prepared in any formulation according to conventional methods. The composition may be formulated, for example, as an oral dosage form (e.g., powder, tablet, capsule, syrup, pill, and granule), or parenteral formulations (e.g., an injection formulation). The composition may also be formulated as a systemic formulation or as a topical formulation.

In the methods described herein, as noted above, the ROCK inhibitor, the immunogenic cell death-inducing chemotherapeutics, and/or the cell population may be administered simultaneously or sequentially. In addition, the ROCK inhibitor, the immunogenic cell death-inducing chemotherapeutics, and/or the cell population may be administered systemically or topically. The desired dosage of the ROCK inhibitor, the immunogenic apoptosis anticancer agent, and/or the cell population varies depending on the condition and the weight of the patient, the severity of the disease, the drug form, the route of administration and the interval of administration, but it can be appropriately selected by those skilled in the art. Such dosages may range, for example, from about 0.001 mg/kg to about 100 mg/kg, from about 0.01 mg/kg to about 10 mg/kg, or from about 0.1 mg/kg to about 1 mg/kg. The administration may be performed once a day, multiple times per day, once a week, once every two weeks, once every three weeks, once every four weeks or once a year.

In another aspect of the present invention, provided is a method for treating a cancer patient, comprising: administering therapeutically effective amount of a ROCK inhibitor and an immune checkpoint inhibitor to the cancer patient.

According to the method, the ROCK inhibitor may be a ROCK2 specific inhibitor which may be fasudil, KD-025 {2-(3-(4-((1H-indazol-5-yl)amino)quinazolin-2-yl)phenoxy)-N-isopropylacet-amide)}, BA-1049, Rho kinase inhibitor V {N-(4-(1H-pyrazol-4-yl)phenyl)-2,3-dihydrobenzo[b][1,4]dioxine-2-carboxamide}, SR3677, or LYC-53976.

According to the method, the ROCK inhibitor may be a ROCK1/2 pan-inhibitor which may be ripasudil, RKI-1447, Y-27632, GSK429286A, Y-30141, thiazovivin, GSK180736A, GSK269962A, Netrasudil, Y-39983, ZInC00881524, Yf-356{(+)-(R)-4-(1-Aminoethyl)-N-(4-pyridyl) benzamide}, Rho kinase inhibitor IV {(S)-(+)-2-Methyl-4-glycyl-1-(4-methylisoquinolinyl-5-sulfonyl) homopiperazine, H-1152}, Rho kinase inhibitor II {N-(4-Pyridyl)-N′-(2,4,6-trichlorophenyl)urea}, SB772077B, Rho kinase inhibitor III {(3-(4-Pyridyl)-1H-indole)}, K-115, HA1100, rho statin, CCG-1423 {N-(2-(4-Chloroanilino)-1-methyl-2-oxoethoxy)-3,5-bis(tri-fluoromethyl)benzamide}, cethrin (VX-210), BA-210, BA-1042, BA-1043, BA-1044, BA-1050, BA-1051, BA-1076, BA-215, BA-285, BA-1037, Ki-23095, or AT13148.

According to the method, the immune checkpoint inhibitor may be a PD-1/PD-L1 interaction inhibitor or a CTLA-4/B7-1/B7-2 interaction inhibitor.

According to the method, the PD-1/PD-L1 interaction inhibitor may be pembrolizumab, nivolumab, atezolizumab or avelumab.

According to the method, the CTLA-4/B7-1/B7-2 interaction inhibitor may be ipilimumab.

The above method may further comprise administering an immunogenic cell death-inducing chemotherapeutic and/or applying photodynamic therapy or radiotherapy, as discussed above.

According to the method, the immunogenic cell death-inducing chemotherapeutics may be an anthracycline-type anticancer agent, cetuximab, paclitaxel, bleomycin, cyclophosphamide, mitoxantrone or oxaliplatin.

According to the method, the anthracycline-type anticancer agent may be daunorubicin, doxorubicin, epirubicin, idarubicin, pixantrone, sabarubicin, or valrubicin.

According to any of the methods, the photodynamic therapy may be applied by administering a photosensitizer and irradiating light capable of activating the photosensitizer.

According to any of the methods, the cancer may be breast cancer, skin cancer, head and neck cancer, pancreatic cancer, lung cancer, colon cancer, colorectal cancer, stomach cancer, ovarian cancer, prostate cancer, bladder cancer, urethral cancer, liver cancer, kidney cancer, papillary carcinoma, melanoma, brain spinal cancer, brain cancer, thymoma, mesothelioma, esophageal cancer, biliary tract cancer, testicular cancer, germ cell tumor, thyroid cancer, parathyroid cancer, cervical cancer, endometrial cancer, lymphoma, myelodysplastic syndromes (MDS), myelofibrosis, acute leukemia, chronic leukemia, multiple myeloma, Hodgkin's disease, endocrine cancer, or sarcoma.

### EXAMPLES

Hereinafter, the present invention will be described in more detail with reference to Examples. However, these examples are intended to illustrate one or more embodiments and the scope of the present invention is not limited to these examples.

### Example 1: Effect of ROCK Inhibitors on Phagocytic and Dendritic Cell Phagocytosis

1-1: Phagocytosis Function Analysis

To determine whether an anti-cancer immune response is enhanced by ROCK inhibitors, the effects of ROCK inhibitors on macrophages and dendritic cells were analyzed.

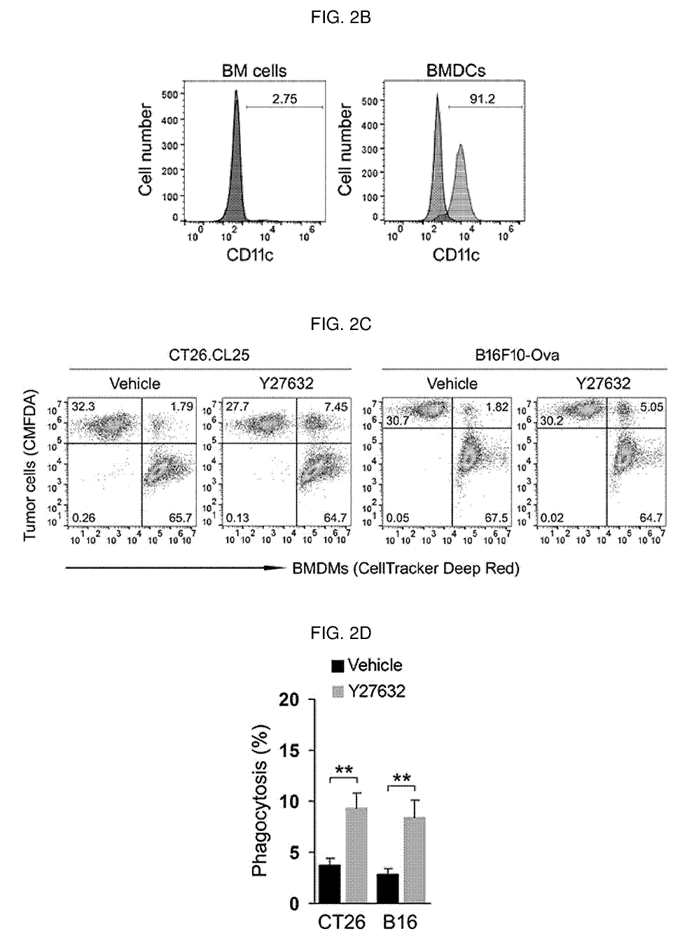

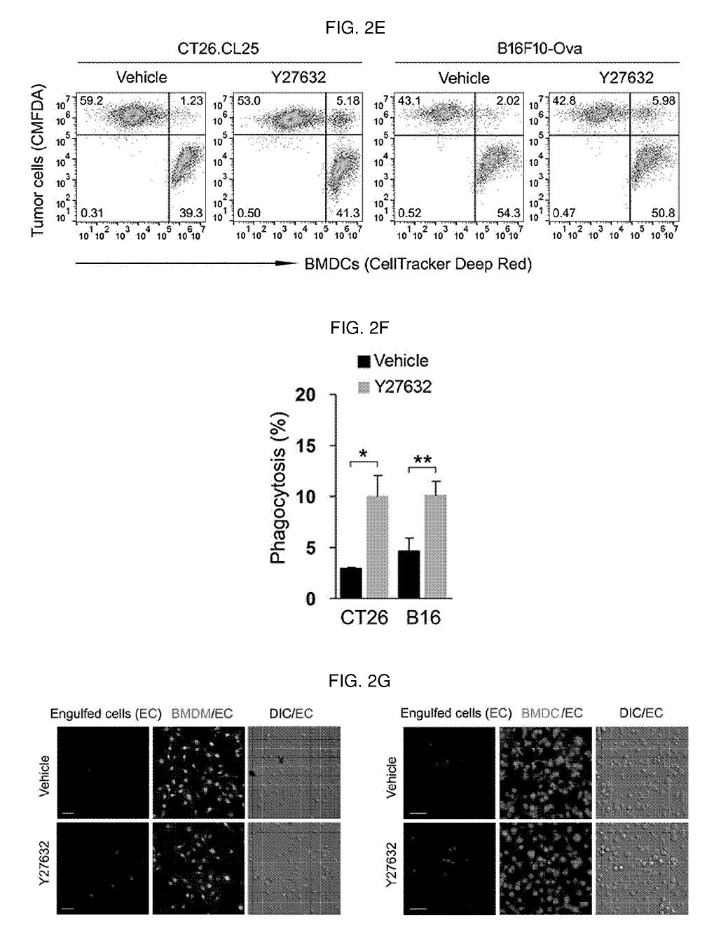

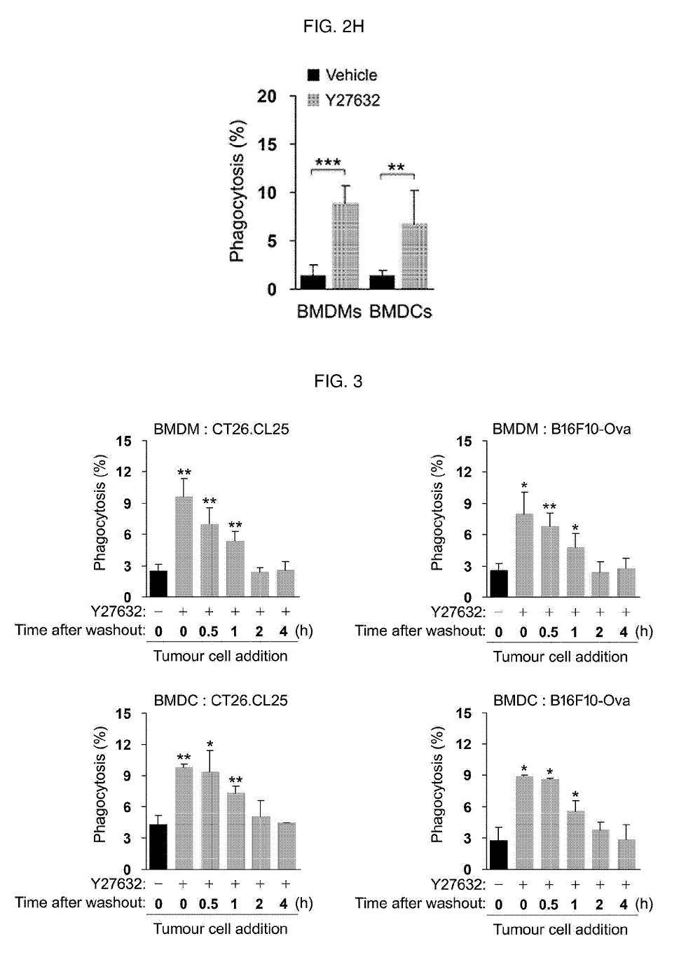

Specifically, the present inventors prepared bone marrow-derived macrophages (BMDMs) differentiated from excised bone marrow of mice using M-CSF and bone marrow-derived dendritic cells (BMDCs) differentiated from excised bone marrow using Flt3L. The degree of differentiation was confirmed by flow cytometry analysis using antibodies specifically binding to F4/80 (BMDM marker) and CD11c (BMDC marker), respectively (FIGS. 2A and 2B).

The prepared BMDMs and BMDCs were stained with 1 μM CellTracker Deep Red (Thermo Fisher Scientific, USA). 30 μM of Y27632 (Abcam, USA) or 30 μM of fasudil (Selleck Chemicals, USA) was added to each of 15×10⁴ cells/ml of the stained macrophages and dendritic cells and incubated at 37° C. for 1 hour. As a negative control, macrophages or dendritic cells that were not incubated with Y27632 and fasudil were used.

Bone marrow-derived macrophages or dendritic cells prepared in RPMI medium and cancer cell lines stained with 1 μM CellTracker CMFDA (Thermo Fisher Scientific, USA) were co-cultured at about 37° C. for about 2 hours. CT26.CL25, a colon cancer cell line, and B16F10-OVA, a melanoma cell line, were used as the cancer cells.

Thereafter, the percentage of cancer cells was measured by flow cytometry, and the phagocytosis ratio of macrophages and dendritic cells against cancer cells was calculated. The results are shown in FIGS. 2C to 2F (left: cancer cell line CT26.CL25, right: cancer cell line B16F10-OVA).

As shown in FIGS. 2G and 2H, it was confirmed that the ROCK inhibitors Y27632 significantly increased phagocytic ability of macrophages and dendritic cells against cancer cells.

In order to more accurately analyze the phagocytosis of macrophages and dendritic cells against cancer cells according to the treatment of the ROCK inhibitor, the present inventors analyzed phagocytosis of the BMDMs and BMDCs against CT26.CL25 colon cancer cells using the pHrodo-SE dye, which changes in fluorescence depending on pH. When phagocytic cells were stained with pHrodo-SE, the pH of the cancer cells decreased and became red. When treated with Y27632 as described above for the above-mentioned phagocytosis response, the phagocytic activities of both the BMDMs and BMDCs against cancer cells were improved.

1-2: Persistence Analysis of Phagocytic Function

The present inventors performed an experiment to confirm how long the phagocytosis of BMDMs and BMDCs against cancer cells due to the ROCK inhibitor is maintained. Specifically, BMDMs and BMDCs were pretreated with Y27632 for 1 hour, then Y27632 was removed, and immediately after 30 minutes, 1 hour, 2 hours, or 4 hours, the phagocytosis against CT26.CL25 and B16F10-Ova was measured using a flow cytometer. The other experimental conditions were the same as in Example 1-1. As shown in FIG. 3, both the BMDMs and BMDCs were confirmed to maintain phagocytosis until 1 hour after the pretreatment with Y27632 (FIG. 3).

1-3: Influence on Phagocytes

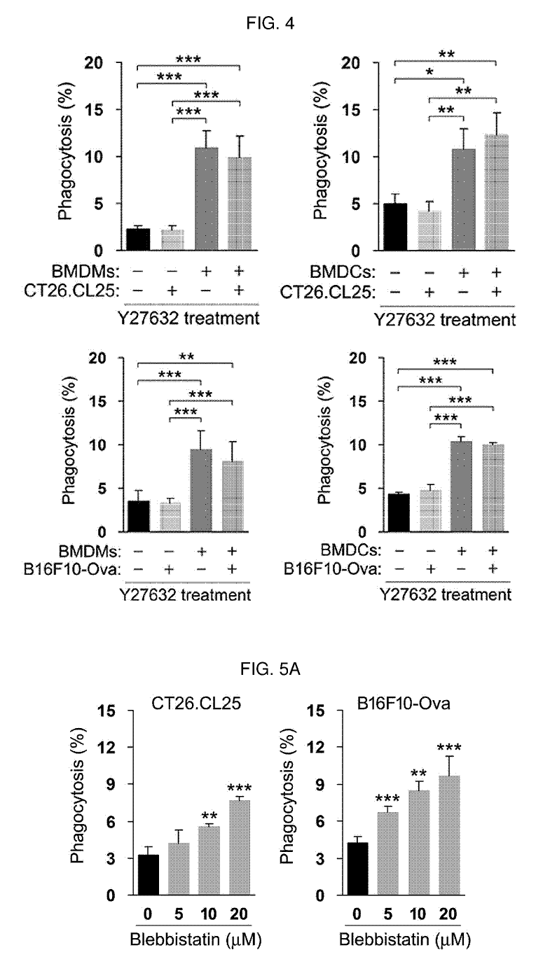

In order to confirm whether the ROCK inhibitor increases phagocytosis against cancer cells by acting on phagocytic cells (BMDMs or BMDCs) themselves, Y27632 was used to pretreat the phagocytic cells or cancer cells for 1 hour, and the phagocytic activity was analyzed using the same procedure described in Example 1-1. As shown in FIG. 4, when the BMDMs or BMDCs were pretreated with Y26732, the phagocytosis against cancer cells was increased, but when cancer cells were pre-treated with Y27632, the phagocytosis against cancer cells was not increased. The above results suggest that Y27632 acts on phagocytic cells rather than cancer cells, thereby increasing phagocytosis of the phagocytic cells against cancer cell (FIG. 4).

1-4: Study on the Mechanism of Phagocytosis of ROCK Inhibitor

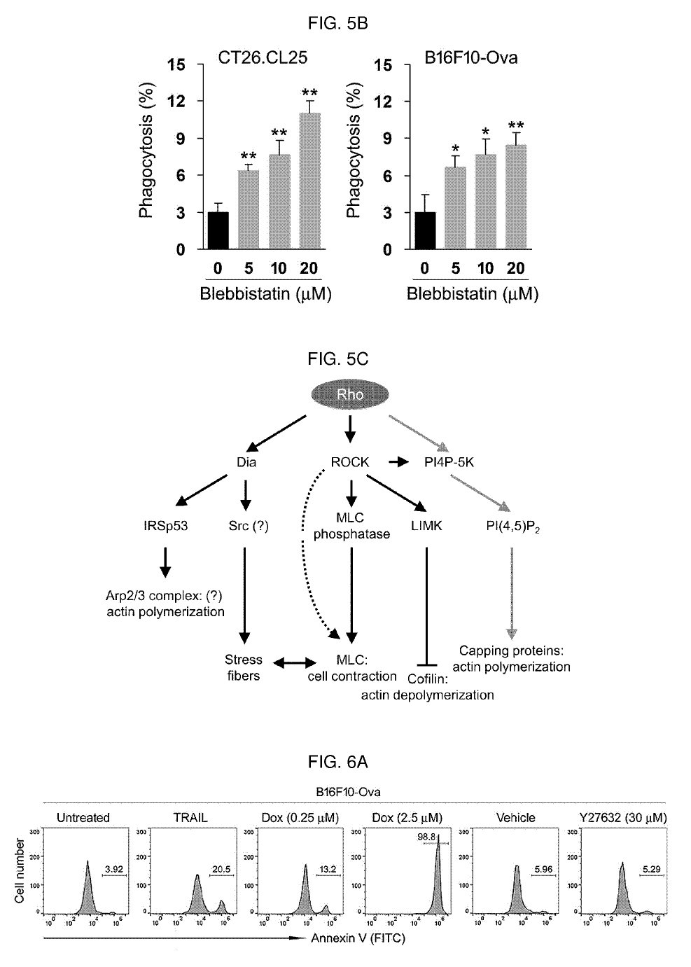

The present inventors identified through the above-mentioned results that Y27632 promotes phagocytic cells against cancer cells by inhibiting ROCK in the phagocytic cells. In order to elucidate a more detailed mechanism related to the increase of phagocytic activity by ROCK inhibitors, the present inventors analyzed phagocytosis of the phagocytic cells against cancer cells using blebbistatin (Sigma-Aldrich, USA), which can inhibit the movement of myosin light chain, which exist downstream of ROCK signaling pathway, according to the method described in Example 1-1. As shown in FIGS. 5A (BMDMs) and 5B (BMDCs), it was confirmed that, similarly to Y27632, blebbistatin could significantly enhance the phagocytic action of phagocytic cells against cancer cells, and thus it was confirmed that the ROCK inhibitors promotes phagocytic activity of phagocytic cells against cancer cells in a ROCK-MLC signal pathway-dependent manner (FIG. 5C).

1-5: Possibility of Direct Death of Cancer Cells by ROCK Inhibitor

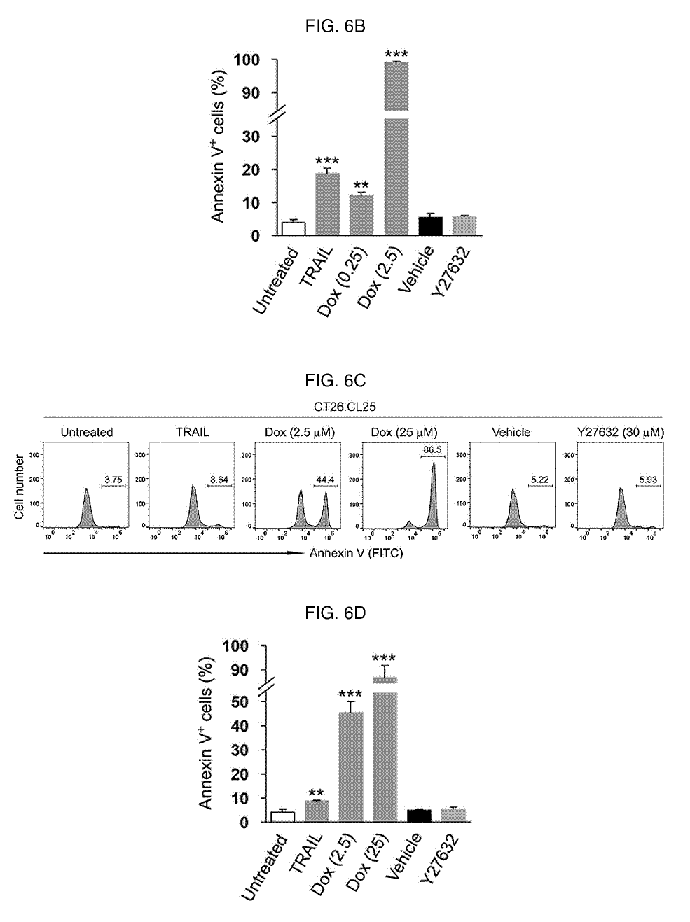

The present inventors investigated whether the ROCK inhibitor directly induced the death of cancer cells and increased the phagocytosis of the phagocytic cells against cancer cells. Specifically, the present inventors treated CT26.CL25 cells and B16F10-Ova cells with 30 μM of the ROCK inhibitor Y27632 for 24 hours, and performed flow cytometry analysis using FITC-anti-Annexin V antibodies (Abcam, USA). On the other hand, cells were treated with 10 μg of TRAIL, 0.25 μM of doxorubicin, 2.5 μM of doxorubicin, or 25 μM of doxorubicin, respectively, for 4 hours, as positive controls. As shown in FIGS. 6A to 6D, it was confirmed that Y27632 was found not to increase the death of cancer cells even though Y27632 was applied for up to 24 hours (FIGS. 6A to 6D).

1-6: Analysis of Cancer Cell Phagocytosis Through Animal Experiments

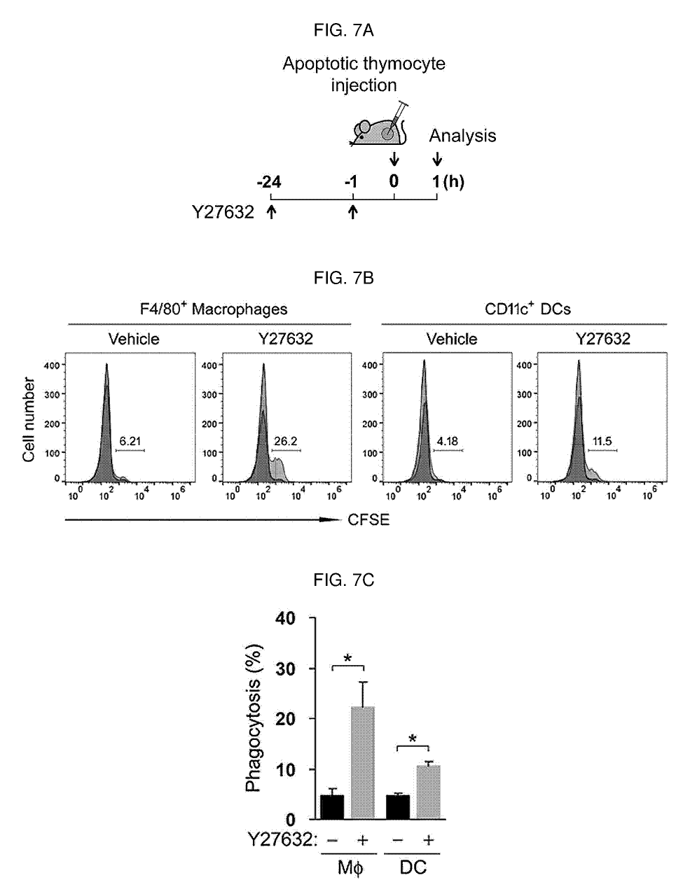

The present inventors investigated whether the phagocytosis caused by the ROCK inhibitor could be enhanced not only in vitro but also in vivo. For this purpose, thymocytes stained with CFSE (carboxyfluorescein succinimidyl ester, Thermo Fisher Scientific, Inc., USA) and in which apoptosis was induced by 1 μM dexamethasone were injected to the blood vessel of mice who were pre-injected with 10 mg/kg of Y27632 intraperitoneally. After 1 hour, the spleen was extracted from the mice and the spleen was single celled. The phagocytic activities of macrophages (F4/80⁺ cells) and dendritic cells (CD11c⁺ cells) against apoptotic thymocytes were analyzed (FIGS. 7B and 7C). As shown in FIGS. 7b and 7c, it was confirmed that both macrophages and dendritic cells showed significantly enhanced phagocytosis against apoptotic thymocytes due to Y27632 (FIGS. 7B and 7C).

### Example 2: Analysis of Cancer Treatment Effect of ROCK Inhibitor in a Tumor Animal Model

2-1: Anticancer Assay for Orthotopic Tumor Model

CT26.CL25 cancer cells (1×10⁶) expressing β-galactosidase were subcutaneously injected into the left back of a Balb/c wild type mice to generate a tumor model Similarly, an orthotopic tumor model was prepared by subcutaneously injecting melanoma cell line B16F10-Ova cells (5×10⁵) into the left back of C57L/B6 wild-type mice to induce cancer.

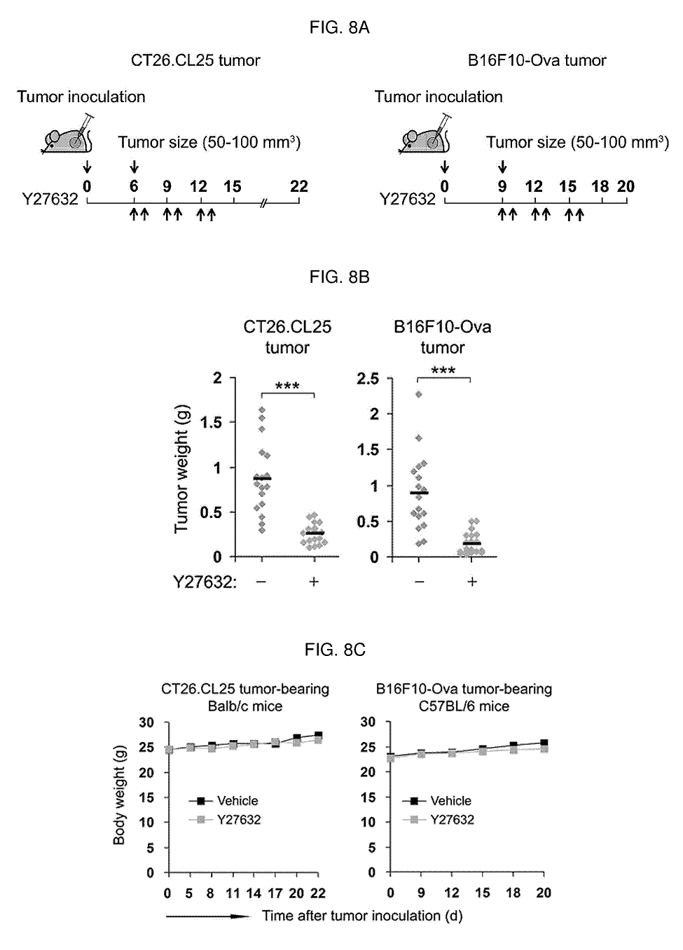

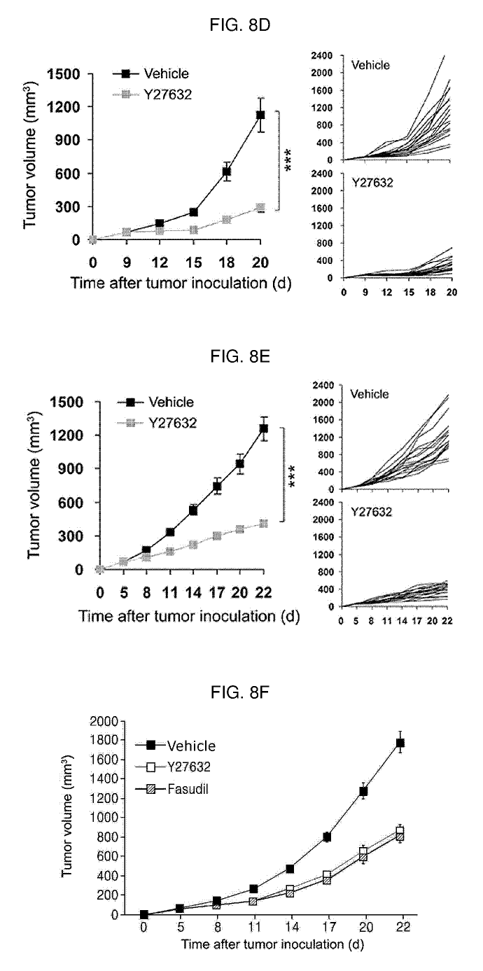

In the case of the CT26.CL25 cancer cell line, 6 days after the injection of the cancer cells, and in the case of B 16F10-Ova, 9 days after the injection of the cancer cells, Y27632 was administered by intravenous injection at a dose of 10 mg/kg as indicated in FIG. 8a. In the case of the CT26.CL25 model, the mice were sacrificed on the 22^{nd} day after the injection of the cancer cells and in the case of the B16F10-Ova cancer model, the mice were sacrificed on the 20^{th} day after the injection of the cancer cells (FIG. 8A). As a negative control, phosphate-buffered saline was administered instead of Y27632.

The tumor tissues of the mice were excised, an ex vivo image was taken and the tumor tissue were weighed (FIG. 8B). In addition, before the mice were sacrificed, changes in body weights were measured at intervals of 3 days from the 6^{th} day of the cancer cell injection in the CT26.CL25 cancer model and at intervals of 3 days from the 9^{th} day in the B16F10-Ova cancer model (FIG. 8c). Length (L) and width (W) of the tumor tissues were measured, and the volume of the tumor tissues over time was calculated using the following formula (FIGS. 8D and 8E):

The volume of tumor tissue (V[mm³])=(L[mm])33 (W[mm])²×0.5.

As shown in FIGS. 8B to 8E, when the ROCK inhibitor Y27632 was administered, the weight and volume of the tumors were significantly decreased in both tumor models, but there was no significant change in the body weight of the mice. Thus, although Y27632 is not toxic to animal models, it has been shown to effectively inhibit cancer growth.

The same procedure as described above was repeated except that CT26 was used instead of the CT26.CL25 colon cancer cell line and fasudil (Selleck Chemicals, USA) at 20 mg/kg of body weight or Y27632 at 10 mg/kg of body weight was administered. The size of the tumor when injected with CT26 cancer cells is shown in FIG. 8F. As shown in FIG. 8F, fasudil, a ROCK inhibitor different from Y27632, inhibited the growth of cancer similarly to Y27632.

2-2: Effect of ROCK Inhibitors in Immune-Deficient Animals

The present inventors investigated the anti-cancer effect of ROCK inhibitors on immunodeficient mice in order to confirm whether ROCK inhibitors act on the anti-cancer immune response.

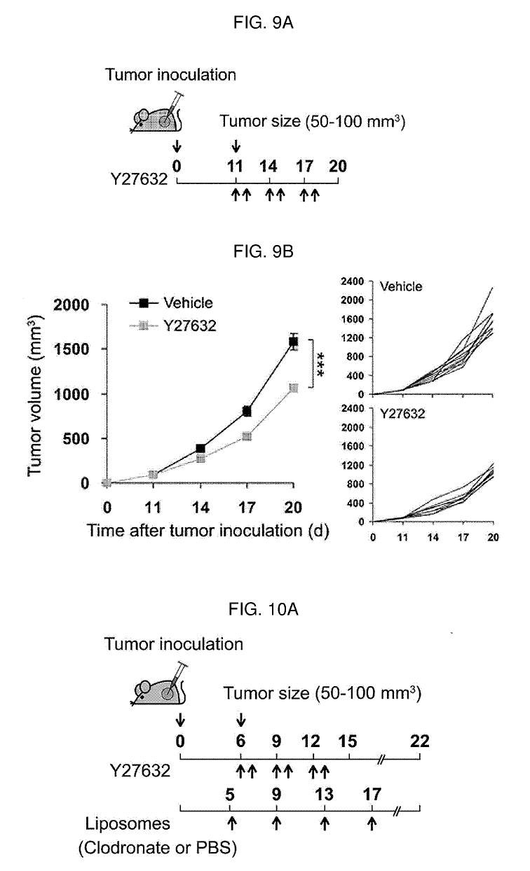

Specifically, immunodeficient nude mice were subcutaneously injected with 1×10⁶ cells of CT26.CL25 cell line in order to induce cancer. After the 11 days from the injection of the cancer cells, tumor size was 50-100 mm³, and Y27632 was administered at a dose of 10 mg/kg continuously for two consecutive days by intravenous injection, followed by a day of rest as a cycle, followed by 3 cycles of 3-day intervals (FIG. 9A). As a negative control group, only phosphate buffered saline (PBS) was administered. The tumor tissues were excised and an ex vivo image was taken. In addition, before the mice were sacrificed, the change in body weight was measured at intervals of 3 days from the 11^{th} day of cancer cell injection in the case of CT26.CL25 cancer model, and the length (L) and width (W) were measured, and the volume of the tumor tissues was measured using the equation described in Example 2-1 (FIG. 9B). As shown in FIG. 9B, in the nude mice lacking immune function, Y27632 showed only a limited anti-cancer effect.

2-3: Anticancer Effect Analysis by ROCK Inhibitor in Depletion of Various Immune Cells

The present inventors analyzed the anticancer activity of the ROCK inhibitor after inducing depletion of immune cells including macrophages, CD8 T cells and CD4 T cells in order to investigate whether the anticancer effect of the ROCK inhibitor is due to action on phagocytic cells such as macrophages and dendritic cells phagocytic cells, an effect of CD8 T cells responsible for cytotoxic immunity, or an effect of CD4 T cells responsible for antibody-dependent immunity.

Specifically, tumor model mice were prepared by subcutaneously injecting 1×10⁶ cells of CT26.CL25 cancer cell line expressing β-galactosidase into wild type mice by subcutaneous injection into the left side or the like.

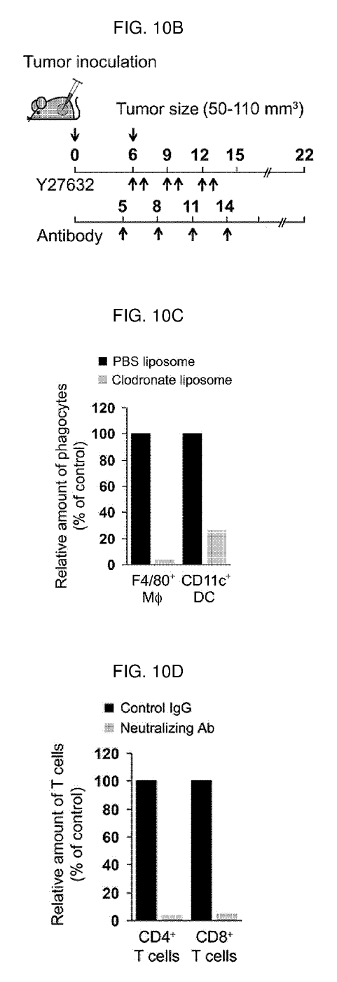

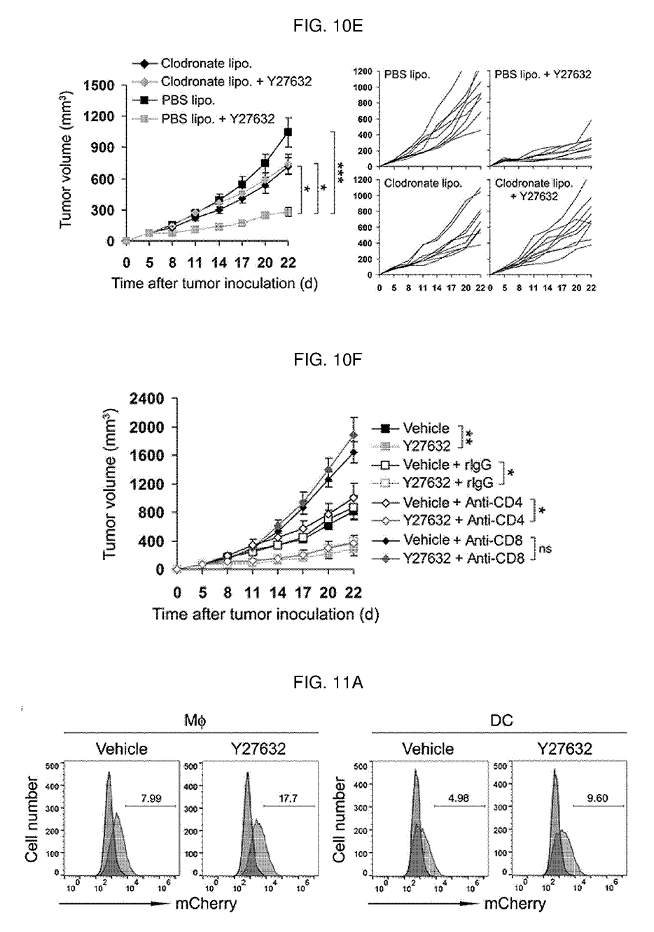

For the depletion of phagocytic cells in the tumor model mice, 200 μl of control liposome (FormuMax Scientific, Inc., USA) or 200 μl of clodronate liposome (FormuMax Scientific, Inc., USA) were injected intraperitoneally 5 days after cancer cell injection, and then 100 μl of the liposomes were intraperitoneally injected at intervals of 4 days until the end of the experiment Similarly, for the depletion of CD8 T cells or CD4 T cells, 200 μg of anti-CD8a antibodies (clone 2.43, bioXcell) or anti-CD4 antibodies (Clone GK 1.5, bioXcell, USA) were intraperitoneally injected (FIGS. 10A and 10B) to the tumor model mice. As a negative control, only the carrier (PBS) or the rat-derived nonspecific antibodies (rIgG, Clone LTF-2, bioXcell, USA) was administered to the tumor model mice. On the other hand, in the case of the clodronate-treated mouse, phagocytic cells were confirmed to be removed as shown in FIG. 10C. It was also confirmed that the anti-CD4 antibody-administered mice and the anti-CD8 antibody-administered mice were deficient in the corresponding T cells (FIG. 10D)

From the 6^{th} day after the injection of the cancer cells, Y27632 was administered at a dose of 10 mg/kg continuously for two consecutive days via intravenous injection, followed by a day of rest as a cycle, followed by 3 cycles of 3-day intervals. Mice were sacrificed 22 days after the injection of cancer cells, the tumor tissues were excised, and ex vivo images were taken, and the tumor tissues were weighed. Before the sacrifice of the mice, the volume of the tumor over time was calculated as described above (FIGS. 10E and 10F). The results showed that the anti-cancer effects of Y27632 disappeared when phagocytic cells were depleted by clodronate or CD8 T cells were depleted by anti-CD8a antibodies, as shown in FIGS. 10E and 10F. Thus, we confirmed that the therapeutic effect of ROCK inhibitors appears to require CD8 T cells. On the other hand, when CD4 T cells were deficient, Y27632 showed an anticancer effect similar to that in normal mice. This suggests that cytotoxic T cell response is an important mechanism for the anticancer effect of Y27632. As a control, a rat-derived nonspecific antibody (rIgG, BioXcell, USA) was used.

2-4: Detection of Cancer Cell Phagocytosis by ROCK Inhibitor

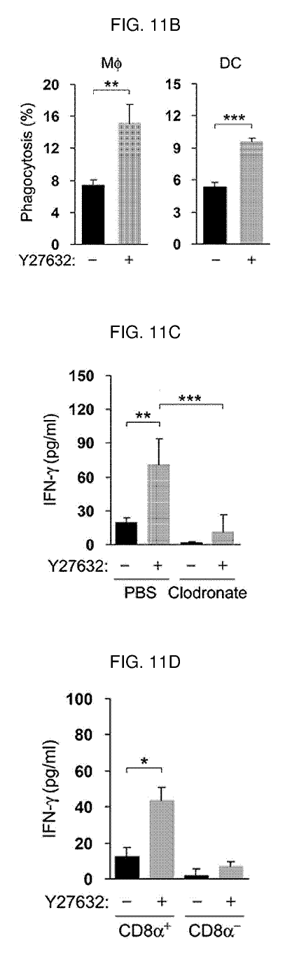

In order to confirm whether or not the ROCK inhibitor Y27632 induces cancer cell phagocytosis in a tumor model, CT26.CL25 colon cancer cells expressing mCherry were prepared and used to create a tumor model by injecting tumor cells into mice, and it was analyzed whether phagocytosis of macrophages and dendritic cells against cancer cells expressing mCherry was increased under the same conditions as in Example 2-3. As shown in FIG. 11A, it was confirmed that the phagocytosis against the cancer cells was increased about two-fold in both macrophages and dendritic cells (FIGS. 11A and 11B).

After draining lymph node was isolated from the tumor model animals, 5 μg/ [symbol] of β-gal peptide (TPHPARIGL, SEQ ID NO: 1, including the naturally processed H-2 Ld limited epitope comprising amino acid residue 876 to 884 of β-galactosidase) which simulates β-gal peptide, a model antigen present in CT26.CL25 cells, or PBS were administered and the expression of IFN-γ was measured by IFN-γ ELISA kit (R&D Systems, Inc., USA). As shown in FIG. 11C, it was confirmed that the IFN-γ level was increased when the ROCK inhibitor was injected in the PBS liposome-treated group, but not in the group treated with the clodronate liposome (FIG. 11C). In addition, it was confirmed by sorting only CD8-positive cells in the draining lymph node, that the amount of IFN-γ was increased in CD8-positive cells, whereas in CD8-negative cells, no difference in INF-γ expression was observed due to administration of Y27632 (FIG. 11D). The above results suggest that Y27632 may act on CD8 T cell-mediated anti-cancer immune response through phagocytosis against cancer cells.

2-5: Anti-Cancer Immunity Memory Induction Assay of ROCK Inhibitor

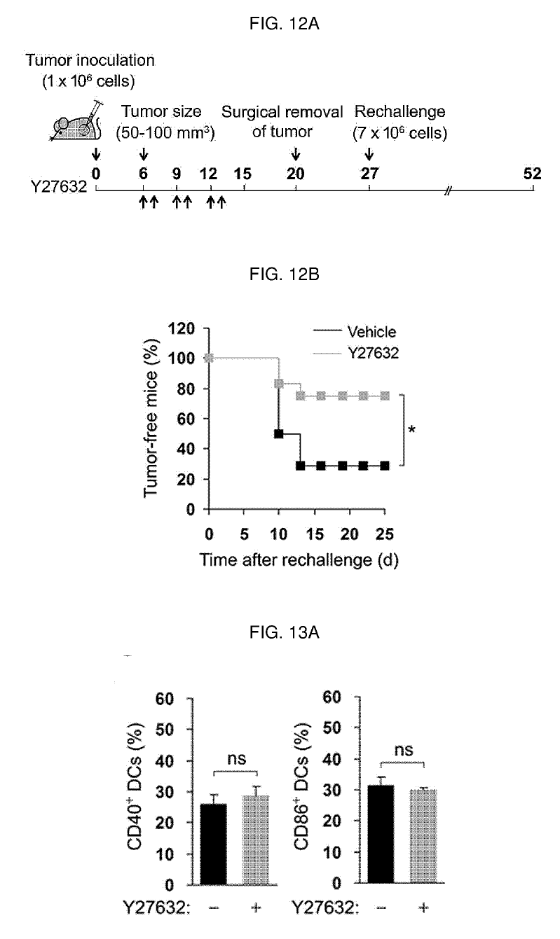

To confirm whether the ROCK inhibitor induces anti-cancer immune memory, CT26.CL25 cells (1×10⁶ cells) were injected into the right flank of Balb/c wild-type mice to induce cancer, and then 10 mg/kg of Y27632 was administered continuously for two consecutive days via intravenous injection, followed by a day of rest as a cycle, followed by 3 cycles of 3-day intervals. After 22^{nd} days from the day of cancer injection, the induced tumor was removed by surgery. One week later, the same cancer cells (7×10⁶ cells) were injected to the other flank and the growth of tumor was investigated (FIG. 12A). As shown in FIG. 12B, it was confirmed that the proportion of tumor-free mice in the ROCK inhibitor-treated group was significantly increased as compared with the control group treated with PBS (FIG. 12B).

2-6: Analysis of the Effect of ROCK Inhibitor in Dendritic Cell Maturation

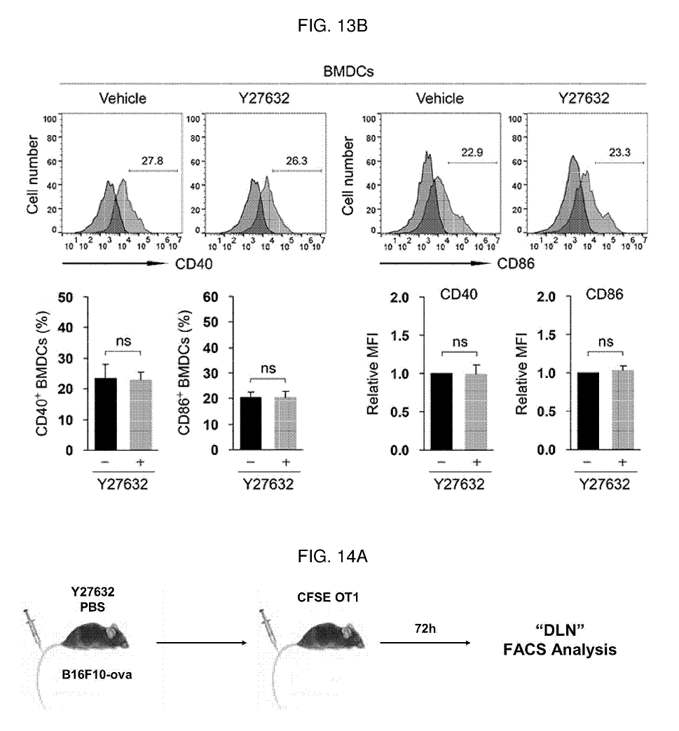

To confirm the effect of the ROCK inhibitor Y27632 on the maturation process of dendritic cells, the inventors administered Y27362 to B16F10-Ova-bearing cancer model animals according to the same administration schedule as in Example 2-1, and the expression of CD40 and CD86, which are dendritic cell maturation markers, was analyzed by flow cytometry. Y27632 did not affect the expression of CD40 and CD86 in dendritic cells (FIG. 13A).

In addition, the present inventors used pretreatment with 30 μM of Y27632 for 1 hour of bone marrow-derived dendritic cells (BMDCs) stained with cell marker CellTracker CMFDA (Thermo Fisher Scientific, Inc., USA) and then removed floating tumor cells by withdrawing removing the supernatant. Twenty hours later, the expression of CD40 and CD86 was analyzed by flow cytometry. It was confirmed that Y27632 alone did not increase the DC maturation in the animal experiment (FIG. 13B).

2-7: Analysis of Cross-Prime Effect of a ROCK Inhibitor on Dendritic Cells

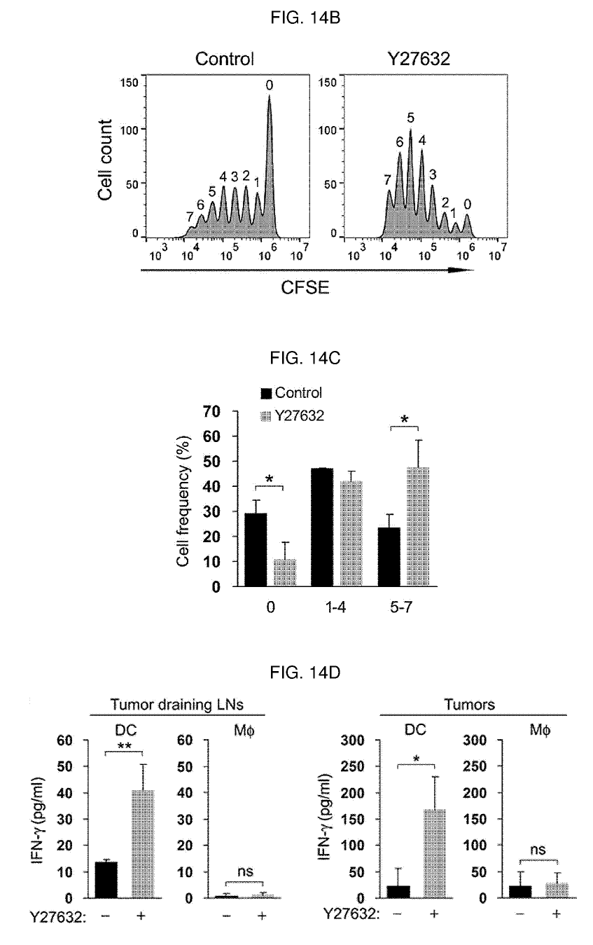

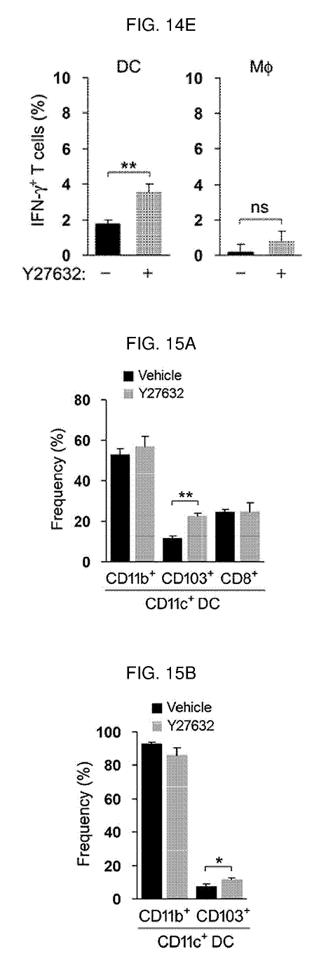

From the results of the above Examples 2-6 showing that ROCK does not affect the maturation of the dendritic cells themselves, the present inventors investigated how ROCK inhibitors have an effect on T cell priming. Specifically, Y27632 was administered to the B16F10-Ova-bearing cancer model mice according to the administration schedule described in Example 2-1, and OT-1 T cells stained with CFSE (carboxyfluorescein succinimidyl ester, Thermo Fisher Scientific, Inc., USA) were injected into the cancer model mice and the degree of proliferation of T cells was confirmed 3 days later (FIG. 14A). It was confirmed that the proliferation of OT-1 T cells was significantly increased in the Y27632 administered group (FIGS. 14B and 14C).

In addition, the expression of IFN-γ in OT-1 T cells co-cultured with macrophages and dendritic cells isolated from the tumor-draining lymph nodes and the tumor tissues in the B16F10-Ova cancer model mice was assessed using IFN-γ ELISA kit (R&D Systems, Inc, USA). It was confirmed that the amount of IFN-γ was increased when the dendritic cells isolated from the tumor-draining lymph nodes and the tumor tissues were co-cultured with OT-1 T cells (FIG. 14D). However, there was no change in the amount of IFN-γ when macrophages and OT-1 T cells were co-cultured. In addition, the proportion of IFN-γ-positive T cells also was increased by co-culture of dendritic cells and OT-1 T cells isolated from tumor-draining lymph nodes, but macrophages isolated from the tumor-draining lymph nodes did not affect the proportion of IFN-γ-positive T cells when co-cultured with OT-1 T cells (FIG. 14E). These results indicate that the ROCK inhibitor alone does not increase the maturation of dendritic cells, but the function of dendritic cells capable of activating T lymphocytes is enhanced.

2-7: Effect of ROCK Inhibitor on CD103 DC

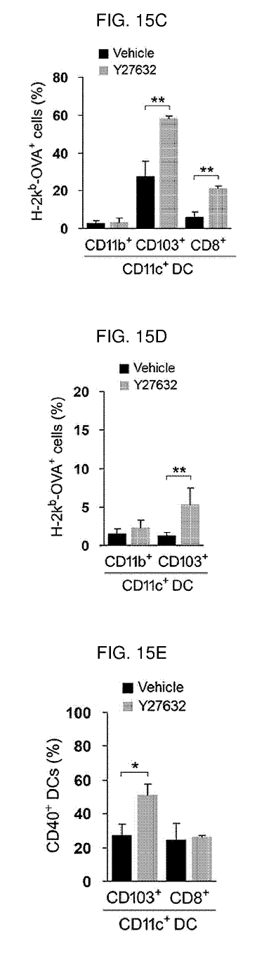

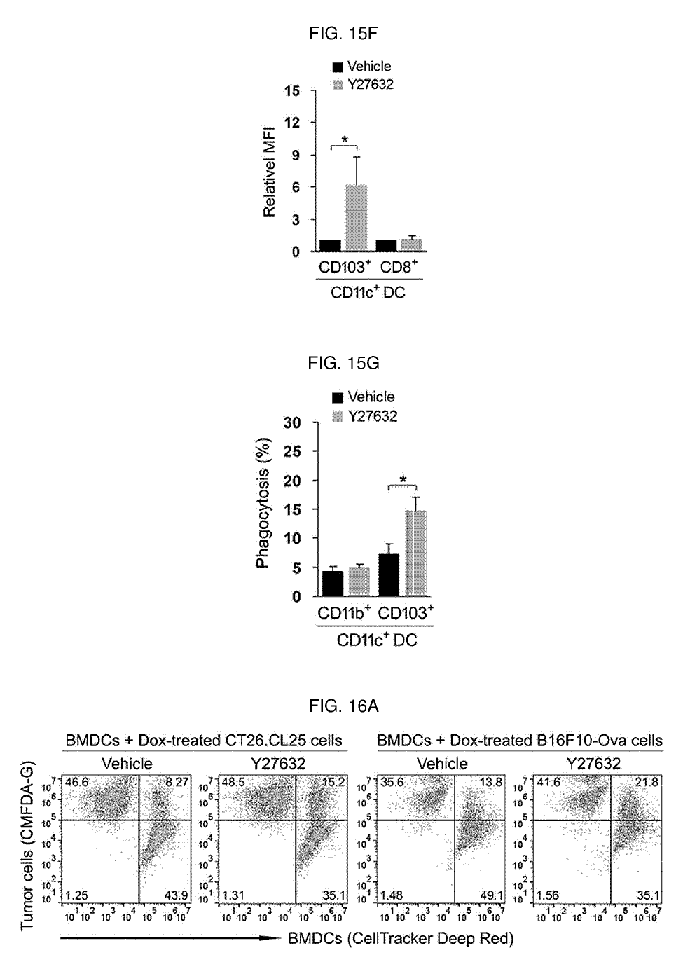

Recently, it has been shown that CD103 (corresponding to CD141 in humans)-positive dendritic cells (CD103 DC) play an important role in cancer cell phagocytosis and T cell anticancer immunity among dendritic cells (Broz et al., Cancer Cell, 26 (5): 638-652, 2014). Therefore, in order to analyze the effect of the ROCK inhibitor on CD103 DC, Y27632 was administered to B16F10-Ova-bearing cancer model mice according to the administration schedule described in the above Example 2-1, tumor tissues and tumor-draining lymph nodes were excised, and then flow cytometry analysis was performed using antibodies specifically binding to dendritic cell markers (CD11b, CD103 and CD8). It was confirmed that the ratio of CD103 DC was increased by Y27632 in both the tumor-draining lymph nodes (FIG. 15A) and the tumor tissues (FIG. 15B). On the other hand, in the case of CD11b-positive and CD8-positive dendritic cells, no change due to administration of Y27632 was observed.

The present inventors analyzed cross presentation activity which is one of functions of dendritic cells via flow cytometry analysis using an antibody capable of detecting a cancer antigen (H-2K^{b}-Ova) capable of activating T cells, through flow cytometry analysis using an antibody (H-2K^{b} monoclonal antibody, Invitrogen, USA). It was confirmed that the cross presentation of CD103 DC was increased by Y27632 in both tumor-draining lymph nodes (FIG. 15C) and tumor tissues (FIG. 15D). In addition, it was confirmed that in the case of tumor-draining lymph nodes, the cross-presentation by Y27632 was also increased in CD8 DCs (FIG. 15C).

Further, the present inventors analyzed CD40, which is one of the markers of dendritic cell function capable of activating T cells and a DC maturation marker of dendritic cells, by flow cytometry analysis using anti-CD40 antibodies. As shown in FIGS. 15E and 15F, it was confirmed that the maturation of CD103 DC was increased by Y27632 in the tumor-draining lymph nodes.

The present inventors excised dendritic cells from tumor tissues and analyzed the phagocytosis of dendritic cells against cancer cells in vitro using the same method as described in Example 1-1. It was confirmed that the CD103 DCs isolated from the mice treated with Y27632 significantly enhanced the phagocytosis against cancer cells than the CD103 DCs isolated from the control mice (FIG. 15G).

These results indicate that Y27632 activates CD103 DCs in dendritic cells and activates T lymphocytes.

### Example 3: Confirmation of Anti-Cancer Immunity Enhancement Effect by the Simultaneous Administration of ROCK Inhibitor and Immunogenicity Cell Death-Inducing Anticancer Drug

3-1: Analysis of Cancer Cell Phagocytosis in Vitro

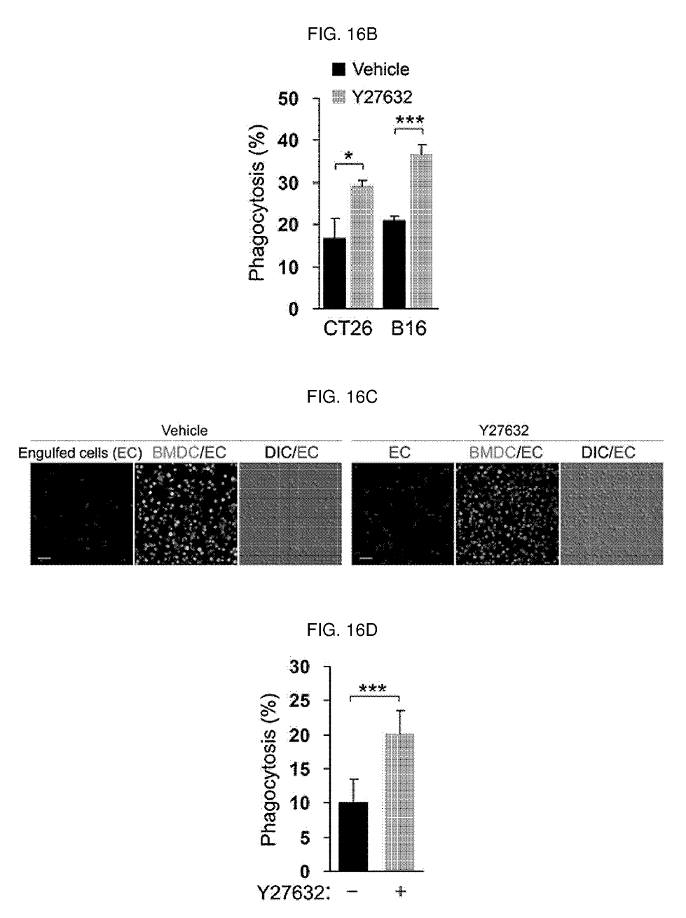

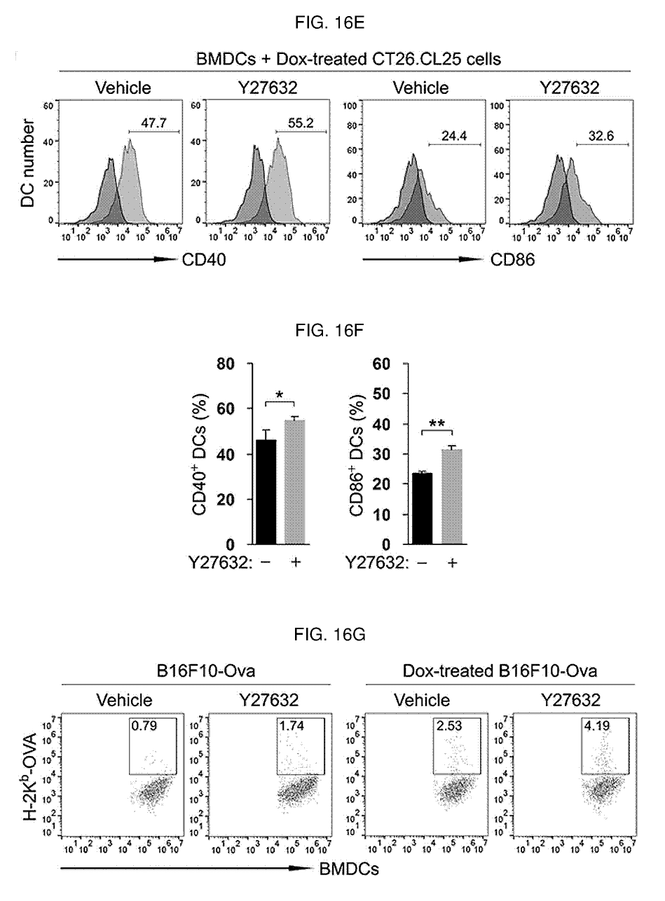

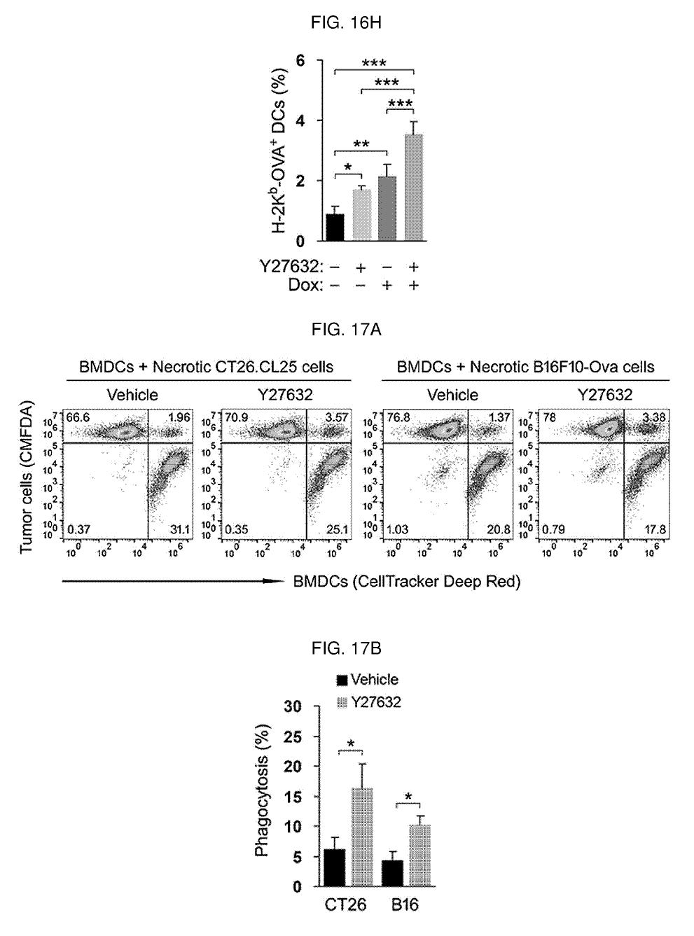

Phagocytic activity of bone marrow-derived dendritic cells (BMDCs) differentiated from bone marrow cells against cancer cells was analyzed by treating the BMDCs with ROCK inhibitor Y27632 (30 μM) for 1 hour and then co-cultivating with CT26.CL25 colon cancer cells pretreated with 25 μM of doxorubicin for 24 hours or B16F10-Ova malignant melanoma cells treated with 30 μM of Y27632 for 1 hour and then treated with 2.5 μM doxorubicin (Sigma-Aldrich, USA) for 24 hours by pHrodo SE (Thermo Fisher Scientific, USA) staining (BMDC:Dox-treated B16F10-Ova=1:1). As shown in FIGS. 16A to 16D, it was confirmed that the phagocytic activity of BMDCs induced by doxorubicin were increased in the Y27632-treated group compared to the control group (FIGS. 16A to 16D).

The present inventors then investigated whether dendritic cell maturation was promoted by the combination treatment of doxorubicin and Y27632. Specifically, 30 μM of Y27632 was used to pretreat bone marrow-derived dendritic cells (BMDC) stained with CellTracker CMFDA (Thermo Fisher Scientific, USA) for 1 hour, and then doxorubicin-treated CT26.CL25 was co-cultured for 4 hours, and floating cancer cells were removed by withdrawing supernatant. The expression level of CD40 and CD86, which are dendritic cell maturation markers, was analyzed by flow cytometry after 20 hours. It was confirmed that dendritic cell maturation was increased in the Y27632-treated group as shown in FIGS. 16e and 16F.

Furthermore, the inventors investigated whether the cross presentation of dendritic cells was promoted by the combination treatment of doxorubicin and Y27632. To this end, the present inventors used 30 μM of Y27632 to pretreat bone marrow-derived dendritic cells (BMDC) stained with CellTracker CMFDA (Thermo Fisher Scientific, USA) for 1 hour, followed by treatment with doxorubicin treated B16F10-Ova cells or doxorubicin untreated B16F10-Ova cells. After the co-culture, floating cancer cells were removed by withdrawing supernatant. Cross presentation activity, which is one of functions of dendritic cells, was analyzed via flow cytometry analysis using an antibody capable of detect a cancer antigen (H-2K^{b}-Ova) capable of activating T cells, through flow cytometry analysis using an antibody (H-2K^{b} monoclonal antibody, Invitrogen, USA). As shown in FIGS. 16G and 16H, Y27632 and doxorubicin (an immunogenic cell death-inducing chemotherapeutic) increased the cross-presentation, and when immunogenic apoptosis of cancer cells was induced using the immunogenic cell death-inducing chemotherapeutic, followed by treatment with Y27632, the cross presentation was increased the most. This suggests that it could be most effective to administer the ROCK inhibitor at certain intervals after inducing immunogenic apoptosis in cancer cells by pretreating with an immunogenic cell death-inducing chemotherapeutic.

3-2: Influence of ROCK Inhibitors on Phagocytosis on Various Apoptotic Cells

From the results of Example 3-1, the present inventors performed the same experiment as Example 3-1 with varying types of cell death, that is, inducing various types of cell death using necrosis-induced cancer cells and a non-immunogenic cell death-inducing chemotherapeutics (cisplatin) in order to confirm whether the enhancement of the phagocytosis of dendritic cells against cancer cells according to the treatment with the ROCK inhibitor is a phenomenon caused by immunogenic cell death induction by doxorubicin.

Specifically, bone marrow-derived dendritic cells (BMDCs) differentiated from bone marrow cells were stained with CellTracker Deep Red (Thermo Fisher Scientific, USA) and 30 μM of ROCK inhibitor, Y27632, was used as pretreatment for 1 hour after incubation with necrotic CT26.CL25 colon cancer cells or B16F10-Ova malignant melanoma cells stained with CellTracker CMFDA (Thermo Fisher Scientific, USA), the degree of phagocytosis against cancer cells was analyzed by flow cytometry. At this time, necrosis of cancer cells was induced by heating in a water bath set at 55° C. for 30 minutes.

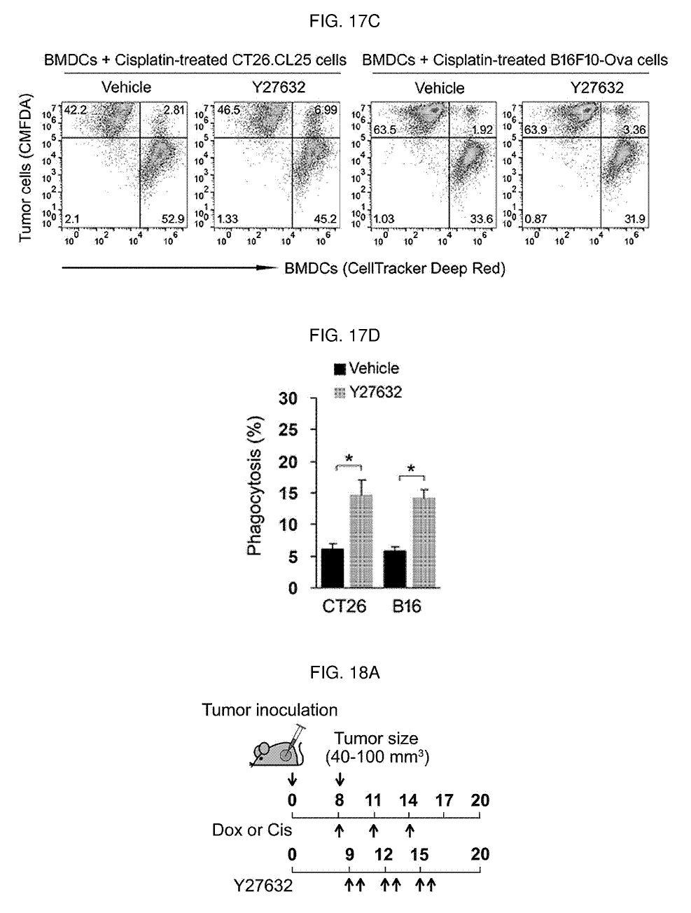

As shown in FIGS. 17A to 17D, phagocytosis of BMDCs against non-immunogenic apoptosis-induced cancer cells by cisplatin (Sigma-Aldrich, USA) and necrosis-induced cancer cells was increased similarly to that by doxorubicin treatment (FIGS. 17A to 17D).

3-3: Anticancer Effect of Combined Administration of Y27632 and Doxorubicin in a Tumor Animal Model

From the above experimental results, the present inventors investigated whether the actual immunogenic cell death-inducing chemotherapeutics can be administered in combination with the ROCK inhibitor to enhance the anti-cancer immune response.

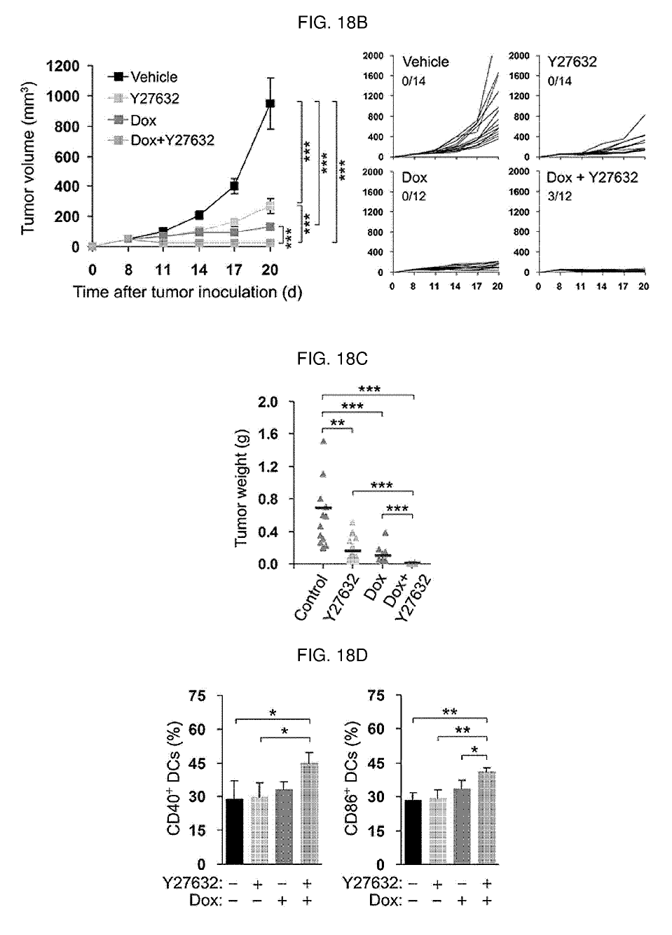

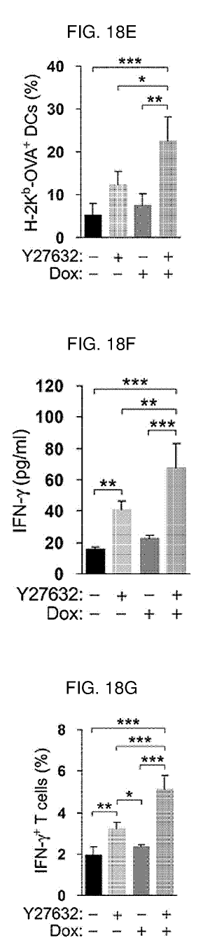

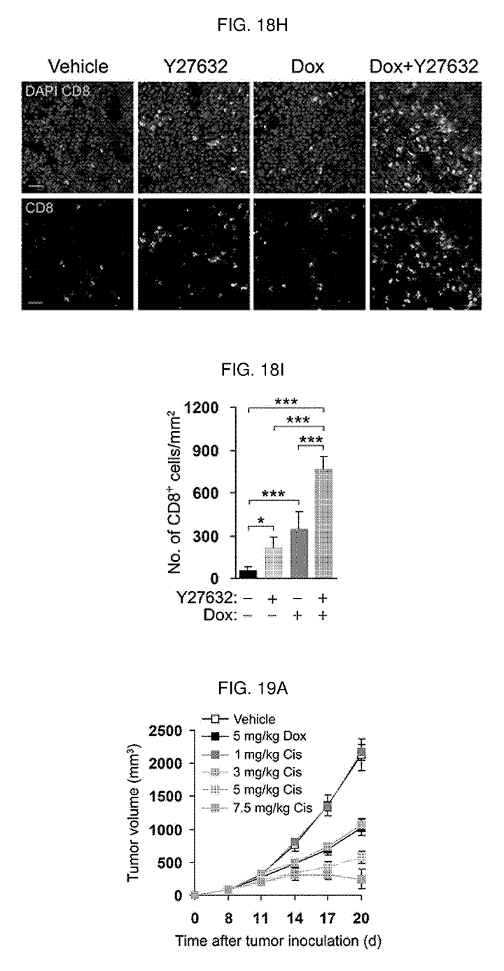

Specifically, tumor suppression was examined by administering 5 mg/kg of doxorubicin and 10 mg/kg of Y27632 alone or in combination to B16F10-Ova-bearing tumor model mice prepared as in Example 2-1. Doxorubicin was administered three times at intervals of 3 days from the 8^{th} day after the injection of cancer cells, and Y27632 was administered at a dose of 10 mg/kg continuously for two consecutive days via intravenous injection followed by a day of rest as a cycle, followed by 3 cycles of 3-day intervals (FIG. 18A). The mice were sacrificed on the 20^{th} day after the injection of cancer cells, the tumor tissue was excised, and ex vivo images were taken and the tumor tissues were weighed. Before the sacrifice of the mice, the tumor volumes over time were calculated as described above using calipers at intervals of 3 days from the 8^{th} day after the injection of the cancer cells.

As shown in FIGS. 18B and 18C, tumor size was significantly decreased in the combination group treated with doxorubicin and the ROCK inhibitor Y27632 compared to the Y27632-only group and the doxorubicin-only group, after 20 days from tumor cell injection. Complete regression of the tumor was not observed in the single administration group, but complete regression of the tumor was confirmed in three of 12 mice in the combination administration group (FIG. 18B).

The present inventors analyzed a series of immunological induction processes occurring after the phagocytosis against cancer cells or cancer-specific antigens in order to analyze the mechanism of the combination therapy for the anti-cancer immune response. Specifically, in order to confirm the effect of the combination therapy on the dendritic cell maturation, Y27632 and doxorubicin were administered to B16F10-Ova-bearing cancer model mice as described above, and then tumor-draining lymph nodes were isolated and the expression of CD40 and CD86, which are dendritic cell maturation markers, was analyzed by flow cytometry analysis. Although Y27632 and doxorubicin monotherapy showed no effect on dendritic cell maturation, it was confirmed that the expression of CD40 and CD86 was increased in dendritic cells of the mice treated with the combination treatment (FIG. 18D).

Further, in order to confirm the effect of the combined treatment on the cross presentation process to the dendritic cells, the present inventors administered Y27632 and doxorubicin to B16F10-Ova-bearing cancer model mice as described above, isolated the tumor-draining lymph nodes, and then cross presentation activity, which is one of functions of dendritic cells, was analyzed via flow cytometry analysis using an antibody capable of detect a cancer antigen (H-2K^{b}-Ova) capable of activating T cells, through flow cytometry analysis using an antibody (H-2K^{b} monoclonal antibody, Invitrogen, USA). As shown in FIG. 18E, the administration of either Y27632 or doxorubicin alone did not significantly increase the antigenic cross presentation in the dendritic cells, but it was confirmed that the antigenic cross-presentation was increased in the dendritic cells in the mice that received the combination treatment (FIG. 18E)

The present inventors investigated whether the combined administration of Y27632 and doxorubicin induces immunity enhancement through CD8 T cells. For this purpose, dendritic cells isolated from tumor-draining lymph nodes of B16F10-Ova-bearing cancer model mice were co-cultured with OT-1 T cells, and then the amount of IFN-γ was measured by IFN-γ ELISA kit (R&D Systems, USA). It was confirmed that T cell priming was increased when Y27632 alone was administered, and T-cell sensitization was more remarkably increased in dendritic cells when Y27632 was combined with doxorubicin (FIG. 18F). In addition, it was confirmed that the amount of IFN-γ-positive T cells increased when the dendritic cells derived from mice administered with the combined administration were co-cultured with OT-1 T cells (FIG. 18G).

In order to confirm whether CD8⁺ T cells infiltrates into tumor tissues, tumor tissues excised from an experimental animal were sectioned and subjected to fluorescence immunohistochemistry using anti-CD8 antibodies. As shown in FIGS. 18H and 18I, a slight infiltration of T cells into tumor tissues when Y27632 and doxorubicin alone was administered was confirmed, and it was confirmed that infiltration of CD8⁺ T cells was significantly increased by the combined administration treatment (FIGS. 18H and 18I).

These results suggest that the combined administration of a ROCK inhibitor and an immunological apoptotic-inducing anticancer agent is effective in increasing immunity against cancer.

3-4: Effect of Combination Therapy with Non-Immunogenic Apoptosis-Inducing Anticancer Drugs

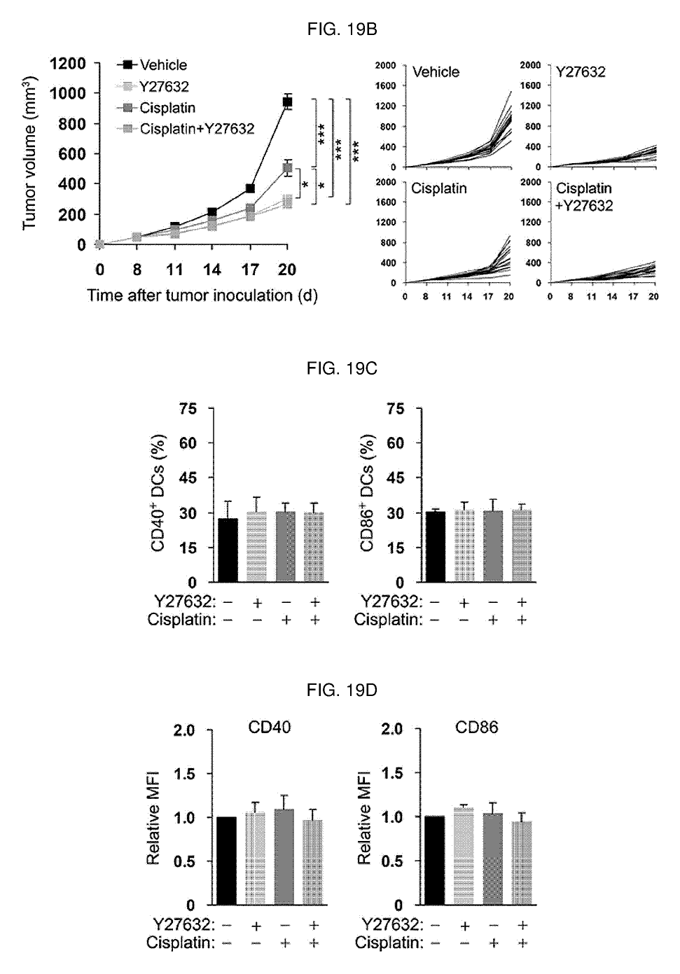

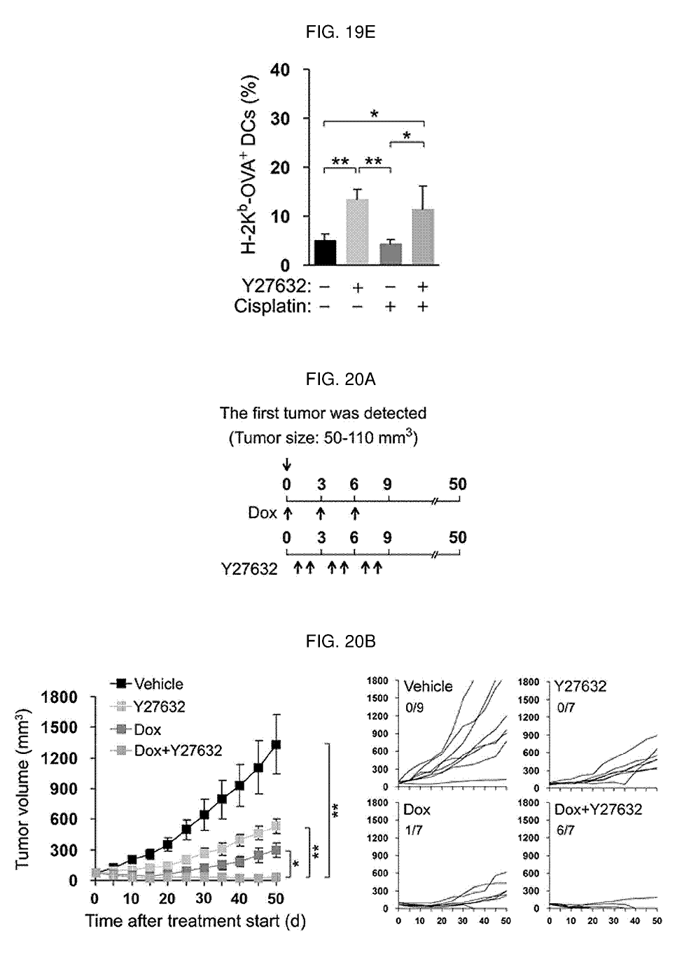

The present inventors performed an experiment in order to confirm whether the administration of cisplatin, which induces non-immunogenic apoptosis unlike doxorubicin, and the ROCK inhibitor Y27632, which induce non-immunogenic cell death, unlike doxorubicin, effectively inhibited the growth of cancer like the combined administration of doxorubicin and Y27632. First, to determine the cisplatin therapeutic concentration, a B16F10-Ova tumor model was constructed in a nude mouse lacking T cell immunity and then an experiment was conducted to confirm the concentration of cisplatin having an anticancer effect similar to that of doxorubicin at 5 mg/kg. As shown in FIG. 19A, 3 mg/kg of cisplatin and 5 mg/kg of doxorubicin showed similar anticancer effects in the animal model. Thus, in order to evaluate the therapeutic effect of cisplatin and the ROCK inhibitor Y27632 in the B16F10-Ova-bearing tumor model, 3 mg/kg of cisplatin was administered according to the administration schedule shown in FIG. 18A.

No significant tumor size reduction was observed in the combination administration of cisplatin and the ROCK inhibitor Y27632 as compared to the Y27632 single administration group and the cisplatin single administration group on the 20^{th} days after tumor cell injection (FIG. 19B).

The present inventors then investigated the effect of the combination of the drugs on the maturation process of dendritic cells. Specifically, the tumor-draining lymph nodes were isolated after administration of Y27632 and/or cisplatin in the B16F10-Ova-bearing cancer model, and the expression level of CD40 and CD86, the dendritic cell maturation markers, was analyzed by flow cytometry. As shown in FIG. 19C and 19D, there was no influence on the maturation of dendritic cells when Y27632 alone, cisplatin alone, or the combination of the two were administered (FIGS. 19C and 19D).

Furthermore, the present inventors investigated the effect of the combination of the drugs on the cross-presentation process of dendritic cells. Specifically, as described above, tumor-draining lymph nodes were isolated after administration of Y27632 and/or cisplatin in the B16F10-Ova-bearing cancer model to analyze a cancer antigen (H-2K^{b}-Ova) displayed on MHC-1 in dendritic cells. For this purpose, a cytometry analysis was performed using an antibody (H-2K^{b} monoclonal antibody, Invitrogen, USA) specific to the cancer antigen to analyze cross-presentation, one of the functions of dendritic cells capable of activating T cells. An increase in the cross-presentation function due to Y27632 was observed as shown in FIG. 19D, but cisplatin had no effect (FIG. 19E).

From the above results, it was confirmed that the ROCK inhibitor significantly increased the immunity against cancer only in combination with an immunogenic apoptosis-inducing anticancer agent.

3-5: Confirmation of Concurrent Administration Effect in Spontaneous Cancer Development Model

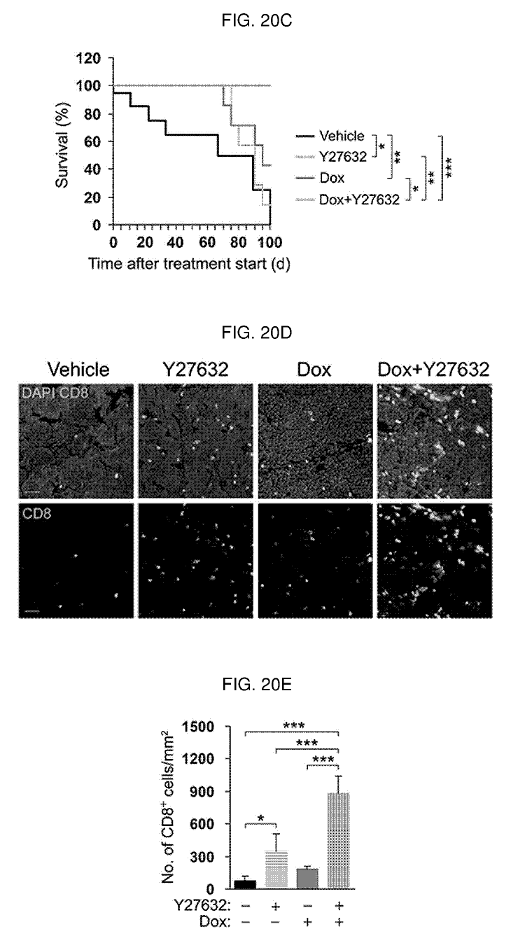

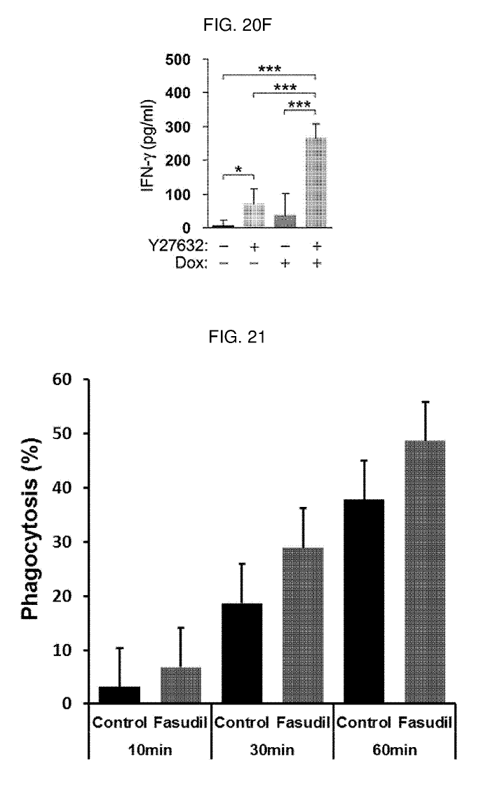

From the above results, the present inventors investigated whether the anti-cancer immunity effect was also induced in the spontaneous cancer development model by the combination administration of the ROCK inhibitor and the immunogenic cell death-inducing chemotherapeutics according to one embodiment of the present invention. Specifically, the present inventors observed MMTV/Neu mice periodically and when the first cancer development was confirmed (considered as 0 day when the tumor size is 50 to 110 mm³), the ROCK inhibitor Y27632 and/or doxorubicin were administered by the administration schedule described in Example 2-1 (FIG. 20A). The survival rate was recorded over up to 100 days. When the size of tumor was over 2,500 mm³, the animal was euthanized (FIG. 20C). As shown in FIG. 20B, tumor growth was significantly inhibited when the combination of drugs was administered as compared to doxorubicin or Y27632 alone. In particular, it was confirmed that the tumors completely disappeared in 6 out of 7 mice in the combination administration group. In addition, as shown in FIG. 20C, survival rates up to 100 days were 100% in the combination administration group.

In addition, the inventors of the present invention investigated the increase of CD8 T cell infiltration into tumor tissues by the combination of the ROCK inhibitor and doxorubicin in the spontaneous cancer development model. Specifically, fluorescence immunohistochemical analysis was performed using anti-CD8 antibodies in the cancer tissues after the end of the experiment. As shown in FIG. 20D, some T cell infiltration was confirmed in the Y27632 and doxorubicin alone groups, and it was confirmed that the amount of CD8⁺ T cells was significantly increased in the combination administration group.

Accordingly, the present inventors investigated whether the combination of the drugs can increase the anti-cancer immunity even in the spontaneous cancer development model. First, in order to confirm whether CD8⁺ T cells were infiltrated into tumor tissues, tumor tissues excised from an experimental animal were sectioned and subjected to fluorescence immunohistochemistry using anti-CD8 antibodies. As shown in FIG. 20e, it was confirmed that the number of CD8⁺ T cells observed in tumor tissues was significantly increased in the Y27632 and doxorubicin combination administration group, as compared with the Y27632 alone group. After completion of the experiment, the present inventors isolated the spleen cells and treated with peptide against rat-Neu, an antigen expressed in the cancer of the spontaneous cancer development model, and after 48 hours, the culture supernatant was collected and IFN-γ was quantified using an ELISA kit (R&D Systems, Inc, USA). As shown in FIGS. 20E and 20F, it was confirmed that the anti-cancer immunity was increased in the Y27632 only group, and the anti-cancer immunity was further increased when the combination treatment with doxorubicin was given.

3-6: Analysis of the Effect of Fasudil and Mitoxantrone

The present inventors investigated whether or not the ROCK2 selective inhibitor fasudil, in addition to the ROCK1/2 pan-inhibitor Y27632, exhibited immunogenic cell death promoting activity equivalent to the above. Bone marrow-derived macrophages (BMDMs) treated with 30 μM of fasudil were co-cultured with B16F10-Ova malignant melanoma cells for 1 hour and the phagocytic activity of the BMDMs against apoptotic cancer cells was analyzed by flow cytometry analysis. In order to induce immunogenic apoptosis, 2.5 μM mitoxantrone (Sigma-Aldrich, USA) was administered with B16F10-Ova cells stained with CellTracker Deep Red (Thermo Fisher Scientific, USA) for 4 hours. The BMDMs stained with CellTracker CMFDA (Thermo Fisher Scientific, USA) and then pretreated with 30 μM of fasudil for 1 hour were co-cultured with the B16F10-Ova cells in DMEM for 30 min at a 1:2 ratio for 10 min, 30 min, and 60 min, respectively. The BMDM treated with fasudil showed a significant increase in the phagocytosis against cancer cells due to immunogenic cell death induced by mitoxantrone, compared with the control group.

### Example 4: Confirmation of Anti-Cancer Immunity Enhancement Effect by Administration of ROCK Inhibitor and Photodynamic Therapy (PDT)

4-1: The Effect of Photodynamic Therapy Against Cancer Cells

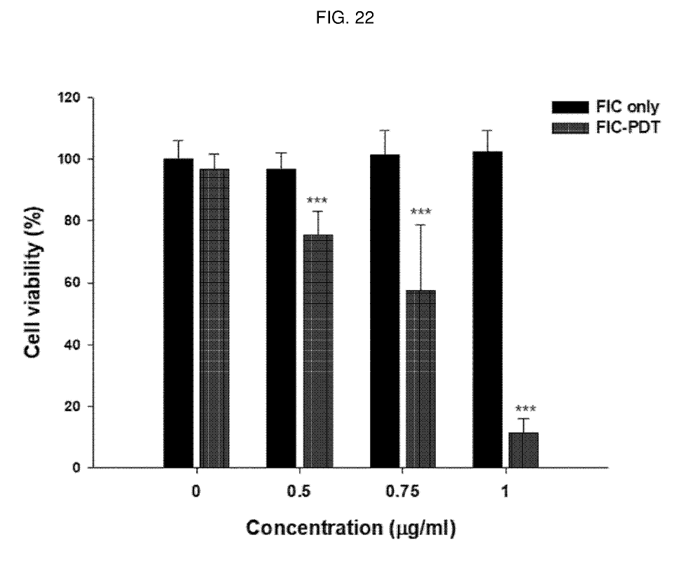

A photodynamic therapy (PDT) was performed by treatment with FIC NPs (0.5, 0.75, and 1 μg/ml), which is a Ce6-based photosensitizer (Lim et al., Small 7(1): 112-118, 2011) of 1×10⁴ cells of B16F10 cancer cell line, known as a mouse melanoma, and irradiating with a light source (LED) at 630-700 nm wavelength for 3 minutes. Cancer cells receive light energy and form reactive oxygen species that can induce cytotoxicity. A CCK assay (Dojindo Molecular Technologies, Inc.) was performed 24 hours later to confirm cell death, and survival rate of cancer cells by PDT was analyzed. As shown in FIG. 22, it was confirmed that as the concentration of photo sensitizer increased, the cancer cell line survived less.

4-2: Analysis of Mechanism of Photodynamic Therapy Against Cancer Cells

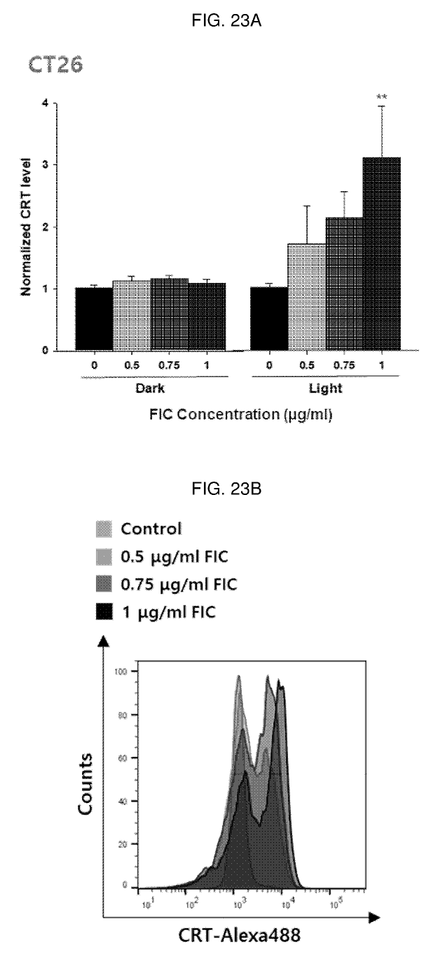

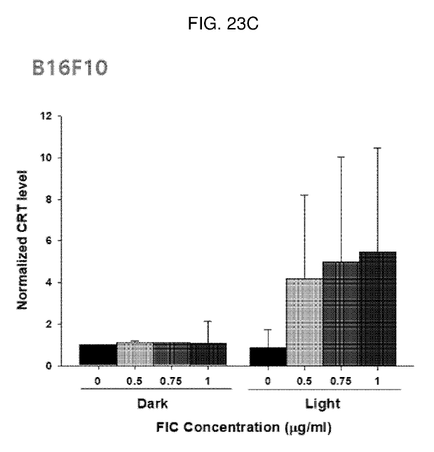

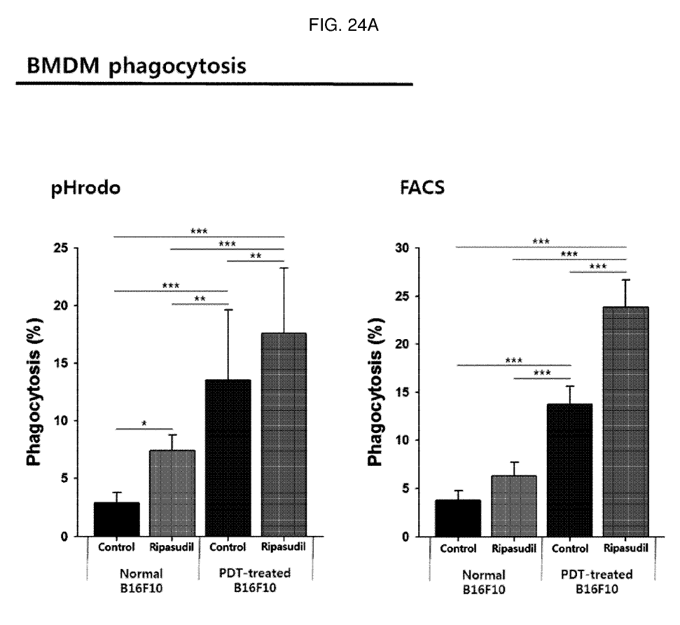

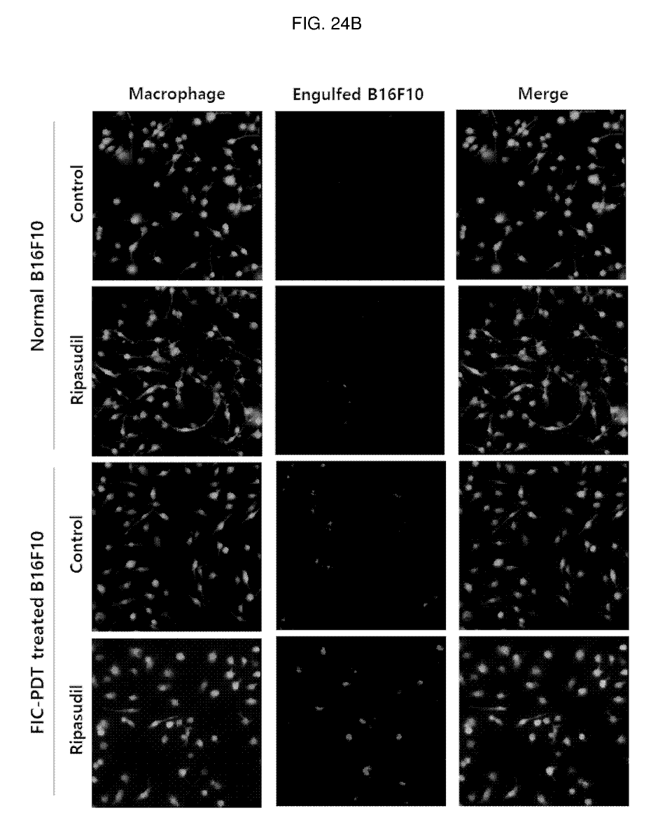

In order to investigate the mechanism of action of the photosensitizer, the present inventors used FIC NPs at the same concentration as in Example 4-1 with the mouse melanoma cell line B16F10 and the mouse colon cancer cell line CT26. At 1 hour after PDT by irradiating with the light source (LED) at 630-700 nm wavelength for 3 minutes, expression level of calreticulin (CRT), which is an “Eat me signal” capable of enhancing phagocytosis of phagocytic cells against cancer cells, was analyzed by flow cytometry using anti-CRT antibodies (abcam). As shown in FIGS. 23A, 23B, and 23C, it was confirmed that CRT expression in the cancer cell line was significantly increased as the concentration of FIC NPs was increased.

4-3: Combination of ROCK Inhibitor Treatment and PDT

The present inventors investigated whether the ROCK inhibitor could further promote the phagocytosis of phagocytic cells against cancer cells in combination with PDT. Particularly, bone marrow-derived macrophages (BMDMs) and bone marrow-derived dendritic cells (BMDCs) were prepared. The prepared cells were stained with 1 μM CellTracker Green (Thermo Fisher Scientific, USA). Macrophages and dendritic cells (15×10⁴ cells/ml) stained with CellTracker Green were treated with 30 μM of ripasudil (Ripa, Selleckchem) and incubated at about 37° C. for 1 hour. As a negative control group, macrophages or dendritic cells not treated with ripasudil were used. The BMDMs or BMDCs prepared in RPMI medium and B16F10 stained with 1 μM of CellTracker Red (Thermo Fisher Scientific, USA, for FACS analysis) or 120 ng/ml of pH rodo SE (Thermo Fisher Scientific, USA, for fluorescence microscopic analysis) were co-cultivated at 37° C. for 30 min. The above cancer cells were subject to PDT treatment or not.

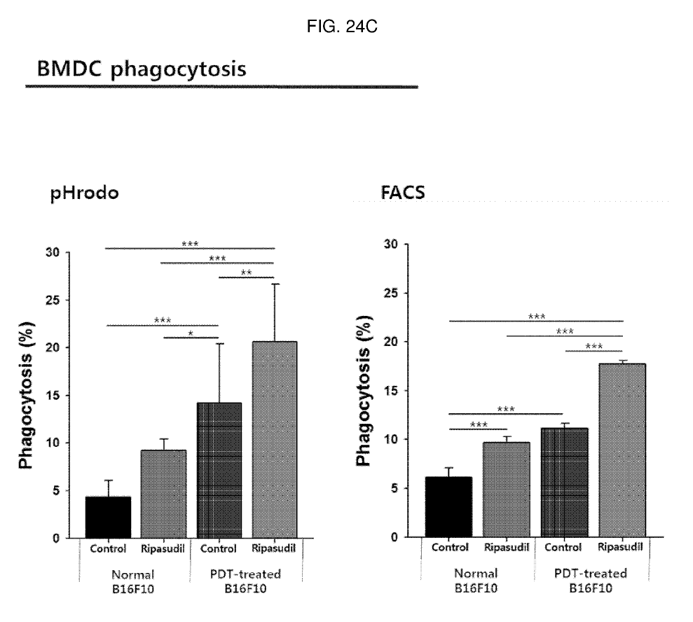

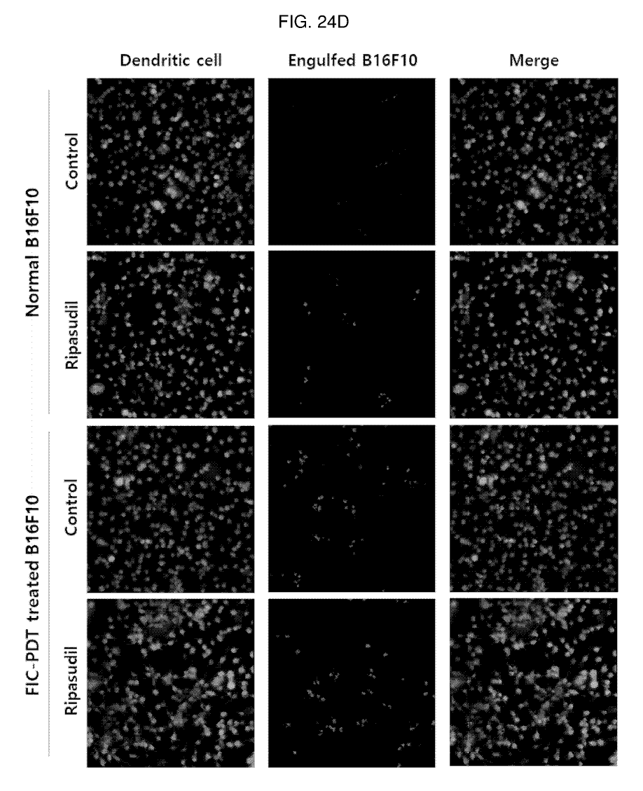

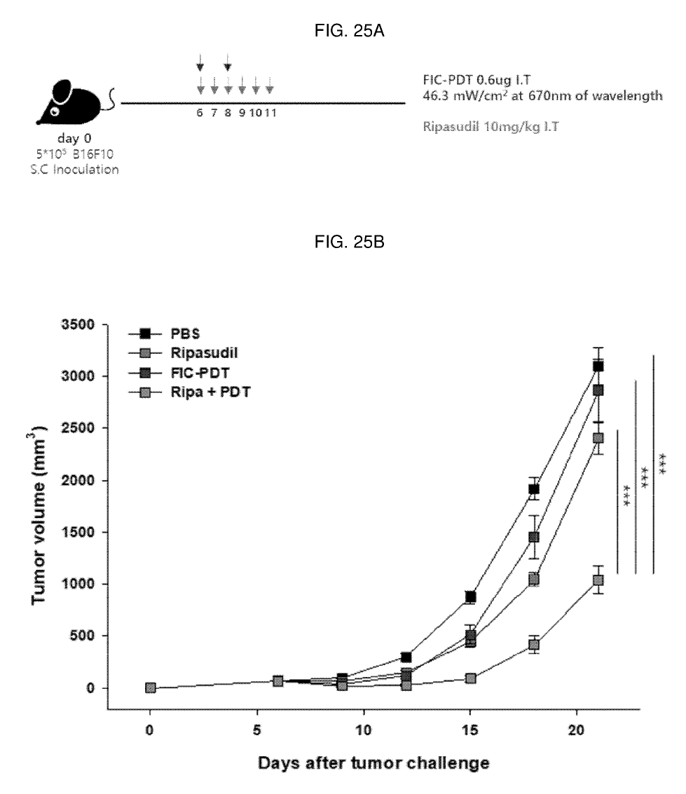

Then, the ratio of engulfed B16F10 to macrophages or dendritic cells was measured by flow cytometry and fluorescence microscopy to calculate the phagocytosis rate. As shown in FIGS. 24A to 24D, it was confirmed that the phagocytic activity of BMDMs and BMDCs treated with the ROCK inhibitor ripasudil was significantly increased in the PDT-treated cancer cells. These results show that the ROCK inhibitor shows a synergistic effect when used in combination with PDT.

4-4: Animal Experiments for the Combination of ROCK Inhibitor and PDT

The inventors of the present invention investigated whether the combination therapy of ROCK inhibitor and PDT was effective in an animal test. Specifically, 5×10⁵ cells of B16F10 cancer cells were injected subcutaneously into C57BL/6 wild-type mice into the left flank and on the 7^{th} day after the injection of cancer cells (day 1 is the date of cancer cell injection), ripasudil was administered at a dose of 10 mg/kg of body weight for 6 consecutive days. On the 7^{th} and 9^{th} days, PDT was performed by irradiating for 30 minutes at 1 hour after injecting 0.6 μg of FIC NPs intratumorally. In the Ripa+PDT group, however, ripasudil was injected 1 hour after PDT treatment on the 7^{th} day when two drugs were administrated simultaneously. As a negative control, PBS was administered instead of ripasudil and FIC NPs.

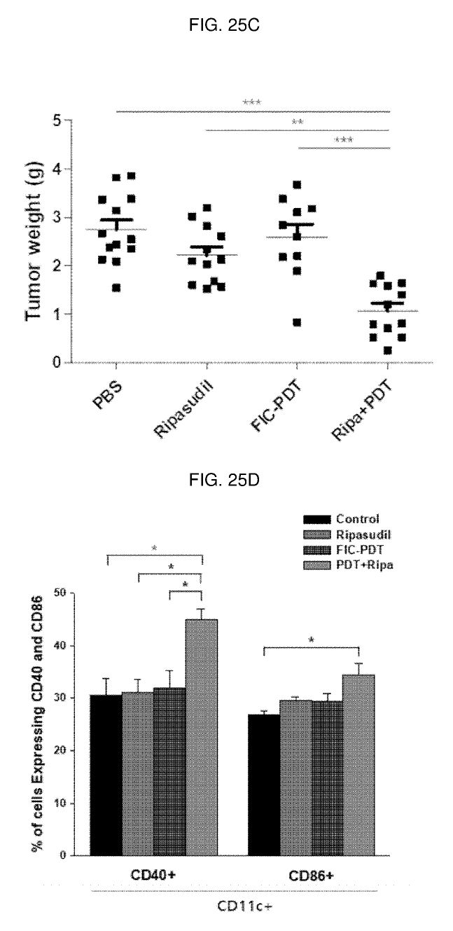

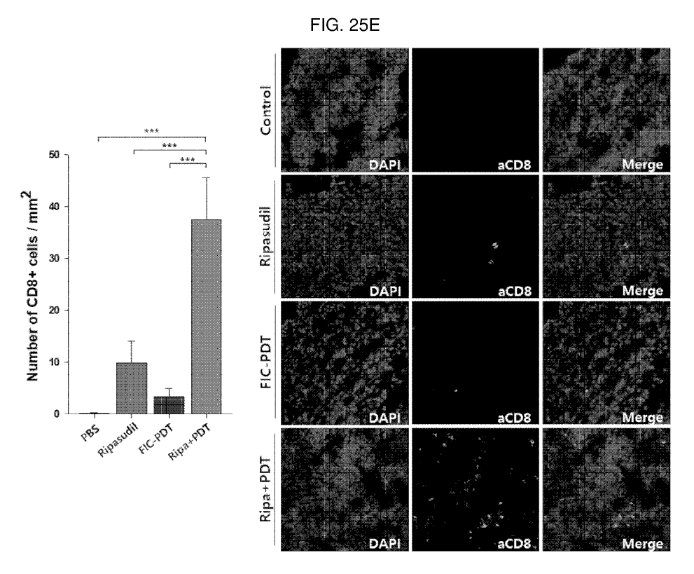

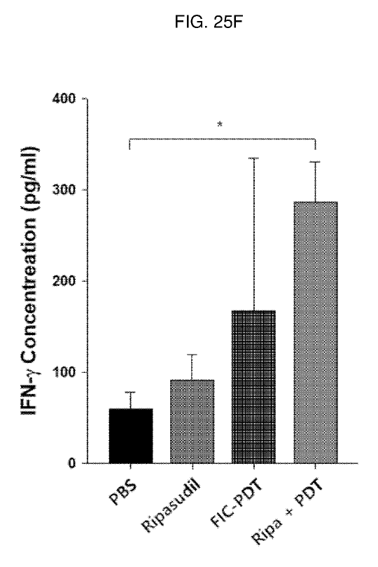

The mice were sacrificed on the 22^{nd} day after cancer cell inoculation, and the tumor tissues were excised and weighed. Before the sacrifice of the mice, the volume of the tumor over time was calculated using calipers at intervals of 3 days from the 7^{th} day after the cancer cell inoculation (FIG. 25B). As shown in FIGS. 25B and 25C, it was confirmed that the anti-cancer effect of Ripa+PDT group was significantly superior to the other groups.

The present inventors conducted an experiment in order to confirm the mechanism of synergistic effect of the combination of ROCK inhibitor and PDT. Particularly, tumor draining lymph nodes were excised from each tumor in the above tumor model animals and made into single cells. And then a flow cytometry analysis was performed using anti-CD11c antibodies (Biolegend) and anti-CD40 antibodies (Biolegend) or anti-CD86 antibodies (Biolegend). CD40 and CD86 are markers indicating the degree of maturation of dendritic cells. As shown in FIG. 25D, a significant increase in CD40 and CD86 was observed upon treatment with ripasudil and PDT. No increased expression of CD40 and CD86 was observed in the ripasudil or PDT only treatment groups, indicating a synergistic effect of combination therapy with ripasudil and PDT.

In addition, the present inventors sought to investigate the effect of ROCK inhibitor and PDT treatment on CD8⁺ T cells in tumor tissues. Particularly, in the above tumor model animals, the tumor tissues were embedded into OCT compound on the 22^{nd} day after the injection of the cancer cells into each group, frozen and sectioned using a vibratome. The cryo-sections were subjected to fluorescence immunohistochemical analysis using anti-CD8a antibodies (BD Pharmingen™). As shown in FIG. 25E, it was confirmed that tumor-specific infiltration of tumor-specific CD8⁺ T cells in the ripasudil and PDT combined treatment groups was increased. In particular, the amount of CD8⁺ T cells infiltrated into the tumor tissues in the combination therapy group was significantly increased compared to the ripasudil or PDT only treatment groups.

Furthermore, the present inventors examined the expression level of interferon-gamma (INF-γ), which is an anticancer cytokine, after combination therapy with ROCK inhibitor and PDT. Particularly, tumor draining lymph nodes were excised from each tumor in the tumor model animals described above and made into single cells. And then, 5×10⁵ cells of lymph nodes and 1000 cells of UV-treated B16F10 cells were co-cultivated in a culture medium containing 100 ng/ml of IL-2 (Pepprotech) capable of activating T cells for 48 hours. Then, the amount of IFN-γ secreted in the culture medium was measured using an INF-γ detection ELISA kit (R&D Systems). As shown in FIG. 25F, it was confirmed that IFN-γ secretion in the ripasudil and PDT combined treatment group was significantly increased.

4-5: Correlation Between ROCK Inhibitor and PDT Combination Therapy and Immune Checkpoint

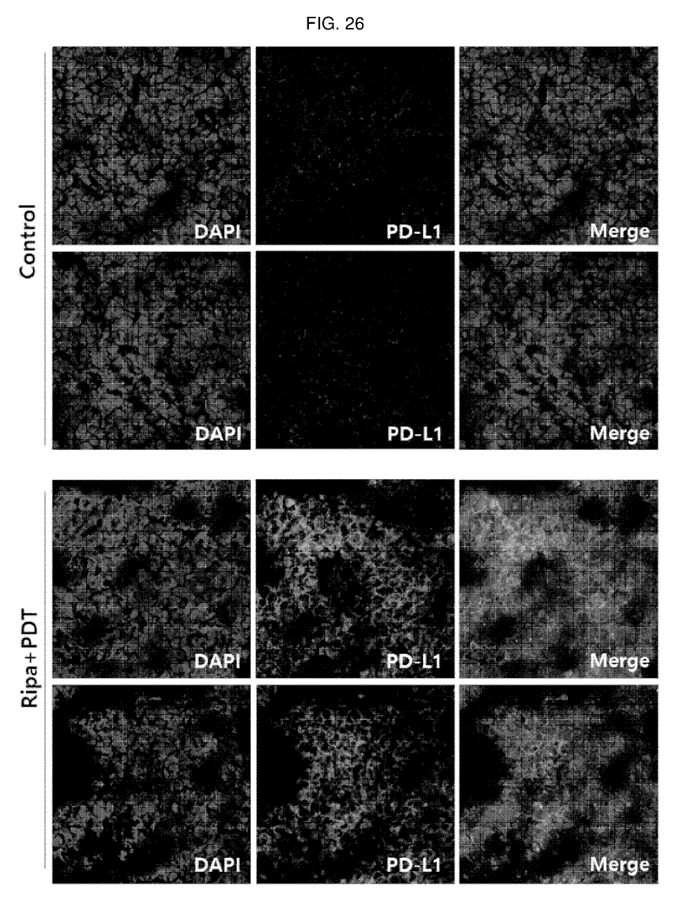

The present inventors investigated how the expression of PD-L1, which is one of the immune checkpoints, changes with treatment with ROCK inhibitor and PDT. Particularly, in each group of the experimental model animals used in Example 4-4, the tumor tissues were excised on the 22^{nd} day after the injection of the cancer cells, embedded in OCT compound, frozen and then sectioned. The cryo-sections were subjected to fluorescence immunohistochemical analysis using anti-PD-L1 antibodies (R&D Systems). It was confirmed that the expression of PD-L1 was increased upon treatment with ripasudil and PDT (FIG. 26). This implies that immune checkpoints are activated in the combination therapy with ROCK inhibitor and PDT, thus suggesting that anticancer treatment targeting an immuno-checkpoint may provide a greater effect.

4-6: Simultaneous Treatment of ROCK Inhibitors, PDT and Anti-PD-L1 Antibodies

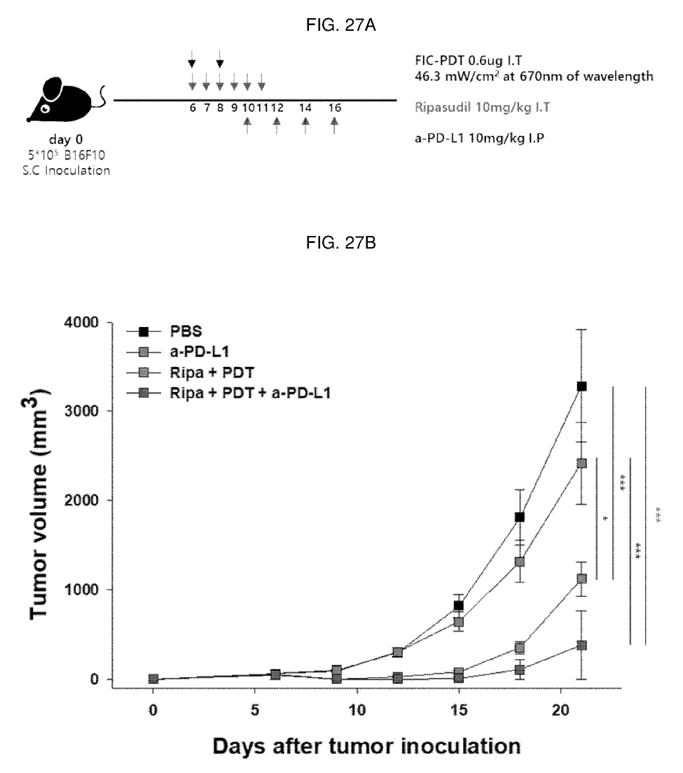

In order to verify the hypothesis derived from the above Example 4-5, the present inventors investigated anti-cancer effects by simultaneously treating with ROCK inhibitor, PDT and anti-PD-L1 antibodies in experimental animals. Particularly, the present inventors injected B16F10 cancer cells (5×10⁵ cells) into C57BL/6 wild-type mice by subcutaneously injecting them into the left flank and on the 7^{th} day after the injection of cancer cells (day 1 is the date of cancer cell injection), ripasudil was administered at a dose of 10 mg/kg of body weight for 6 consecutive days. On the 7^{th} and 9^{th} day, PDT was performed by injecting 0.6 μg FIC NPs intratumorally and irradiating for 30 minutes at 1 hour after the injection of FIC NPs. However, ripasudil was injected 1 hour after PDT treatment on the 7^{th} day in the Ripa+PDT combination treating group. PD-L1 antibodies were intraperitoneally injected 4 times at intervals of 2 days from the 11^{th} day after inoculating cancer cells. When called for by the schedule, anti-PD-L1 antibodies were administered together with ripasudil. The volume of the tumor over time was calculated using a caliper at intervals of 3 days from the 7^{th} day after inoculating cancer cells, as described above (FIG. 27B). As shown in FIG. 27B, it was confirmed that the anti-cancer effect was significantly superior in the group treated with ripasudil, PDT and anti-PD-L1 antibodies simultaneously, as compared to the other groups.

### Example 5: Combination of Immunogenic Cell Death Inducers, ROCK Inhibitor and Anti-PD-L1 Antibodies

5-1: Combined Treatment of Mitoxantrone and Fasudil

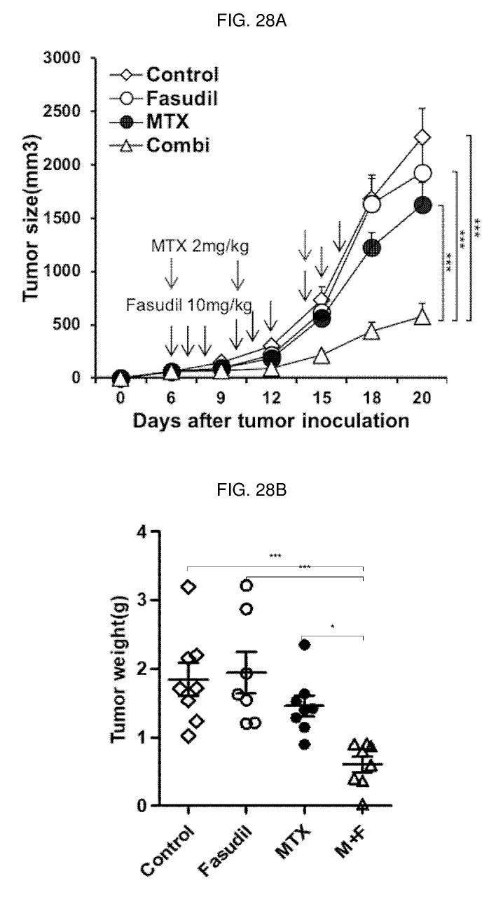

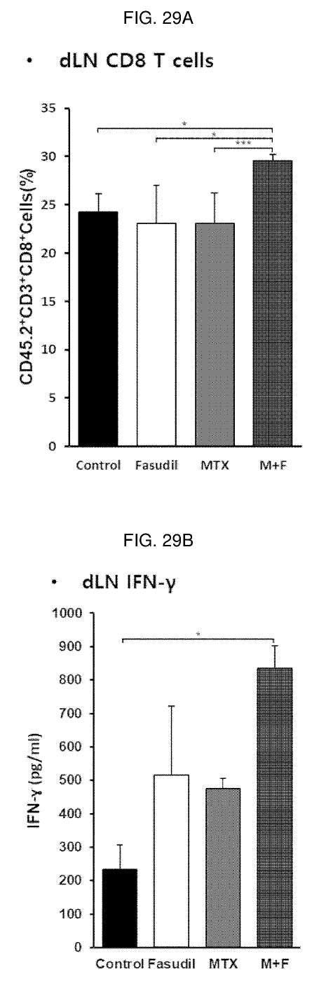

The present inventors also investigated whether mitoxantrone (MTX), another immunogenic cell death inducer, has synergistic anticancer effects when administered in combination with the ROCK inhibitor fasudil. Particularly, 5×10⁵ cells of B16F10 were injected into the C57BL/6 wild-type mice by subcutaneously injecting them to the left flank, and mitoxantrone (Sigma) was injected intravenously to the mice on the 7^{th} day after inoculating cancer cells (day 1 is the date of cancer cell injection) at a dose of 2 mg/kg of body weight at 4-day intervals. Fasudil (Selleckchem) was administered intravenously for 3 consecutive days at a dose of 20 mg/kg of body weight from the 7^{th} day after inoculating cancer cells, and the schedule of administration was repeated three times after a day's rest. In the MTX+fasudil combination treatment group, on the 7^{th}, 11^{th}, and 15^{th} day, when the two drugs were co-administered, the fasudil was injected 4 hours after administering mitoxantrone. As a negative control, PBS was administered instead of mitoxantrone and fasudil. Mice were sacrificed on the 21^{st} day after inoculating cancer cells, and the tumor tissues were excised and weighed. Before the sacrifice of the mice, the volume of the tumor over time was measured using the caliper at intervals of 3 days from the 7^{th} day after inoculating cancer cells, as described above. As shown in FIGS. 28A and 28B, it was confirmed that the antitumor effect in the group simultaneously treated with MTX and fasudil was significantly improved compared to the other groups.

5-2: Analysis of Mechanism of MTX and Fasudil Combined Therapy Against Cancer Cells

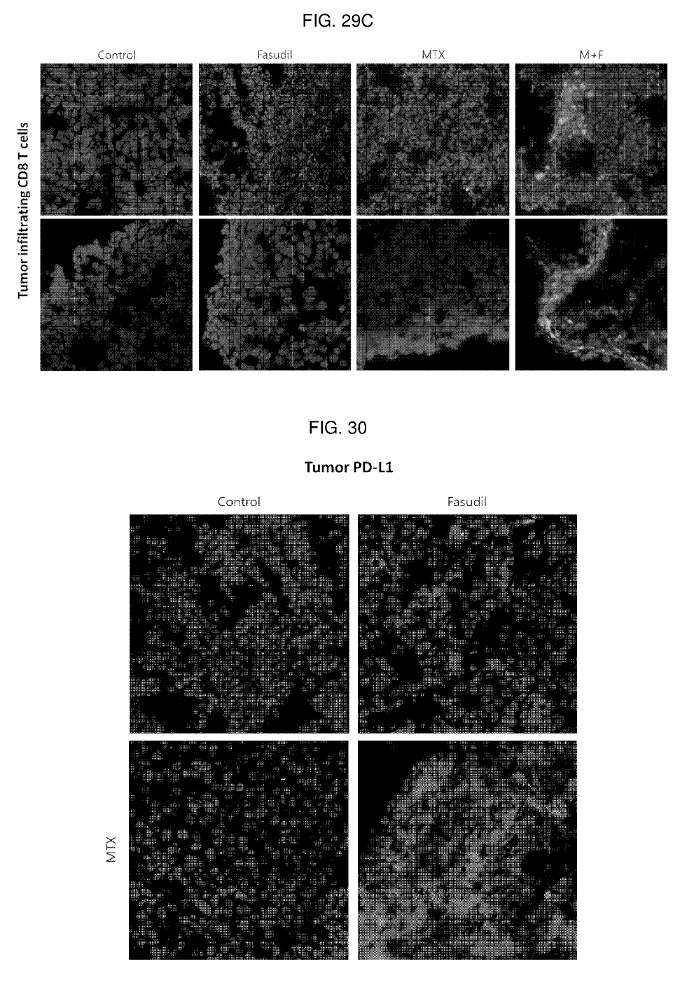

The present inventors sought to investigate the effect of combined treatment of ROCK inhibitor and MTX on CD8⁺ T cells in tumor tissues. Particularly, tumor draining lymph nodes were excised from each tumor in the above tumor model animals and made into single cells. Then a flow cytometry analysis was performed using anti-CD45.2 antibodies (Biolegend), anti-CD3 antibodies (Biolegend) and anti-CD8 antibodies (Biolegend) and analyzed ratio of CD45.2-, CD3-, and CD8-positive cells, namely CD8⁺ T cells in the lymph nodes. As a result, as shown in FIG. 29A, CD8-positive T cells in the MTX+Fasudil combined treatment group were increased significantly compared to the other groups.

In addition, the present inventors examined the secretion of interferon-gamma (INF-γ) in the combination therapy of ROCK inhibitor and MTX. Particularly, the tumor draining lymph nodes were excised from each tumor model animal and made into single cells. Then, 5×10⁵ cells of lymph nodes and 1000 cells of UV-treated B16F10 cells were co-cultured in a culture medium containing 100 ng/ml of IL-2 (Pepprotech) capable of activating T cells for 48 hours. Then, the amount of IFN-γ secreted in the culture medium was measured using an INF-γ detection ELISA kit (R&D Systems). As shown in FIG. 29B, it was confirmed that IFN-γ secretion in the MTX and fasudil combined treatment group was significantly increased.

Further, the tumor tissues were embedded into OCT compound on the 21^{st} day after the inoculation of the cancer cells into each group, frozen, and sectioned using a vibratome. The cryo-sections were subjected to fluorescence immunohistochemical analysis using anti-CD8a antibodies (BD Pharmingen™). As shown in FIG. 29C, it was confirmed that infiltration of tumor-specific CD8⁺ T cells into the tumor in the MTX and fasudil combined treatment group was increased.

5-3: Correlation Between ROCK Inhibitor and MTX Combination Therapy and Immune Checkpoint

The present inventors investigated how the expression of PD-L1, one of the immune checkpoints, changes in the combined therapy of MTX and fasudil. Particularly, in each group of the experimental model animals used in Example 5-2, the tumor tissues were excised on the 21^{st} day after the inoculation of the cancer cells, embedded in OCT compound, frozen and then sectioned. The cryo-sections were subjected to fluorescence immunohistochemical analysis using anti-PD-L1 antibodies (R&D Systems). It was confirmed that the expression of PD-L1 was increased upon treatment with MTX and fasudil (FIG. 30). This implies that immune checkpoints are activated by the combination therapy with ROCK inhibitor and immunogenic cell death inducer, thus suggesting that anticancer treatment targeting an immune checkpoint may provide a greater effect.

5-4: Simultaneous Treatment of ROCK Inhibitors, Immunogenic Cell Death Inducer and Anti-PD-L1 Antibodies

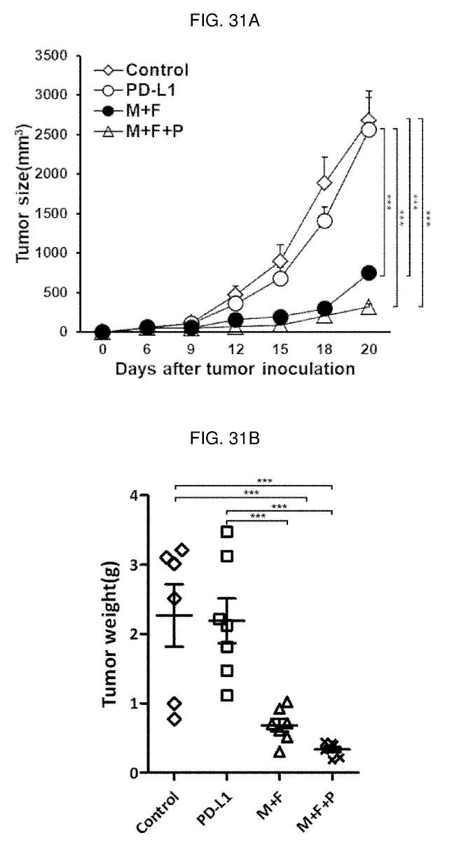

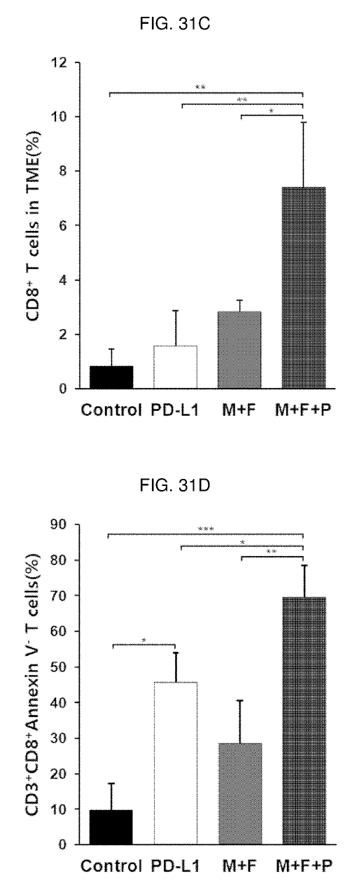

In order to verify the hypothesis derived from the above Example 5-3, the present inventors investigated anti-cancer effects by simultaneously treating with ROCK inhibitor, immunogenic cell death inducer and anti-PD-L1 antibodies in experimental animals. Particularly, the present inventors injected B16F10 cancer cells (5×10⁵ cells) into C57BL/6 wild-type mice by subcutaneously injecting them into the left flank and on the 7^{th} day after the inoculation of cancer cells (day 1 is the date of cancer cell injection), mitoxantrone was administered intravenously at a dose of 2 mg/kg of body weight at 4-day intervals. Fasudil was administered at a dose of 20 mg/kg body weight on the 7^{th} day after the injection of cancer cells for 3 consecutive days and the schedule of administration was repeated three times after a day's rest. In the MTX+fasudil combination treat group, on the 7^{th}, 11^{th}, and 15^{th} day, when the two drugs were co-administered, the Fasudil was injected 4 hours after administering mitoxantrone. Anti-PD-L1 antibodies were intraperitoneally injected 5 times at intervals of 2 days from the 11^{th} day after inoculating cancer cells. When called for by the schedule, anti-PD-L1 antibodies were administered together with fasudil. As a negative control, PBS was administered instead of mitoxantrone, fasudil and anti-PD-L1 antibodies. Mice were sacrificed on the 21^{st} day after inoculating cancer cells, and the tumor tissues were excised and weighed. Before the sacrifice of the mice, the volume of the tumor over time was measured using the caliper at intervals of 3 days from the 7^{th} day after inoculating cancer cells, as described above. As shown in FIGS. 31A and 31B, it was confirmed that the anti-cancer effect was significantly superior in the group treated with MTX, fasudil, and anti-PD-L1 antibodies simultaneously as compared to the other groups.

The present inventors sought to investigate the effect of combined treatment of ROCK inhibitor, MTX and anti-PD-L1 antibodies on CD8⁺ T cells in tumor tissues. Particularly, tumor tissues were excised from each tumor model animal and made into single cells. Then a flow cytometry analysis was performed using anti-CD3 antibodies (Biolegend), anti-CD8 antibodies (Biolegend) and anti-Annexin647 (AdipoGen Life Sciences) and analyzed ratio of CD8⁺ T cell in the tumor tissues. As shown in FIGS. 31C and 31D, the ratio and survival rate of CD8⁺ T cells in the tumor tissue of MTX+fasudil+anti-PD-L1 antibodies combined treatment group were increased significantly compared to the other groups.

What is claimed is:

1. A method for activating dendritic cells in a subject, comprising administering a Rho-related protein kinase (ROCK) inhibitor to the subject.

2. The method according to claim 1, wherein the ROCK inhibitor is a ROCK2 selective inhibitor or a ROCK1/2 pan-inhibitor.

3. The method according to claim 2, wherein the ROCK2 specific inhibitor is selected from fasudil, KD-025 {2-(3-(4-((1H-indazol-5-yl)amino)quinazolin-2-yl)phenoxy)-N-isopropylacet-amide)}, BA-1049, Rho kinase inhibitor V {N-(4-(1H-pyrazol-4-yl)phenyl)-2,3-dihydrobenzo[b][1,4]dioxine-2-carboxamide}, SR3677, and LYC-53976.

4. The method according to claim 2, wherein the ROCK1/2 pan-inhibitor is selected from ripasudil, RKI-1447, Y-27632, GSK429286A, Y-30141, thiazovivin, GSK180736A, GSK269962A, netrasudil, Y-39983, ZInC00881524, Yf-356{(+)-(R)-4-(1-Aminoethyl)-N-(4-pyridyl) benzamide}, Rho kinase inhibitor IV {(S)-(+)-2-Methyl-4-glycyl-1-(4-methylisoquinolinyl-5-sulfonyl) homopiperazine, H-1152}, Rho kinase inhibitor II {N-(4-Pyridyl)-N′-(2,4,6-trichlorophenyl)urea}, SB772077B, Rho kinase inhibitor III {(3-(4-Pyridyl)-1H-indole)}, K-115, HA1100, rhostatin, CCG-1423 {N-(2-(4-Chloroanilino)-1-methyl-2-oxoethoxy)-3,5-bis(tri-fluoromethyl)benzamide}, cethrin (VX-210), BA-210, BA-1042, BA-1043, BA-1044, BA-1050, BA-1051, BA-1076, BA-215, BA-285, BA-1037, Ki-23095, and AT13148.

5. The method according to claim 1, wherein the dendritic cells are cancer-associated dendritic cells.

6. The method according to claim 5, wherein the caner-associated dendritic cells are CD103- or CD141-positive dendritic cells.

7. The method according to claim 1, wherein the ROCK inhibitor is administered via oral administration, intravenous administration, intramuscular administration, intranasal administration, intraperitoneal administration, subcutaneous administration, intradermal administration, intracardiac administration, intraocular administration, intrathecal administration, intraarticular administration, intraarterial administration, sublingual administration, intravaginal administration, intracranial administration or transmucosal administration.

8. The method of claim 1, further comprising
administering an immunogenic cell death-inducing chemotherapeutic to the subject.

9. The method of claim 1, further comprising applying photodynamic therapy or radiation therapy to the subject.

10. The method according to claim 8, wherein the immunogenic cell death-inducing chemotherapeutic is selected from an anthracycline-type anticancer agent, cetuximab, paclitaxel, bleomycin, cyclophosphamide, mitoxantrone and oxaliplatin.

11. The method according to claim 8, wherein the immunogenic cell death-inducing chemotherapeutic is an anthracycline-type anticancer agent selected from daunorubicin, doxorubicin, epirubicin, idarubicin, pixantrone, sabarubicin, and valrubicin.

12. The method according to claim 8, wherein the ROCK inhibitor and the immunogenic cell death-inducing chemotherapeutic are administered simultaneously or sequentially at regular intervals.

13. A method of selectively activating cancer-associated dendritic cells in a cell population comprising dendritic cells or progenitor cells of the dendritic cells, comprising treating the cell population with a ROCK inhibitor.

14. The method according to claim 13, wherein the treating is performed in vitro or in vivo.

15. A method for treating a cancer patient, comprising:
administering (i) a cell population comprising dendritic cells or progenitor cells of the dendritic cells treated with a ROCK inhibitor and (ii) an immunogenic cell death-inducing chemotherapeutic to the cancer patient; or
administering a cell population comprising dendritic cells or progenitor cells of the dendritic cells treated with a ROCK inhibitor and applying a photodynamic therapy or radiation therapy to the cancer patient.

16. The method according to claim 15, wherein the cancer is selected from breast cancer, skin cancer, head and neck cancer, pancreatic cancer, lung cancer, colon cancer, colorectal cancer, stomach cancer, ovarian cancer, prostate cancer, bladder cancer, urethral cancer, liver cancer, kidney cancer, papillary carcinoma, melanoma, brain spinal cancer, brain cancer, thymoma, mesothelioma, esophageal cancer, biliary tract cancer, testicular cancer, germ cell tumor, thyroid cancer, parathyroid cancer, cervical cancer, endometrial cancer, lymphoma, myelodysplastic syndromes (MDS), myelofibrosis, acute leukemia, chronic leukemia, multiple myeloma, Hodgkin's disease, endocrine cancer, and sarcoma

17. The method according to claim 15, wherein the cell population and the immunogenic cell death-inducing chemotherapeutic are administered simultaneously or sequentially at regular intervals

18. The method according to claim 15, wherein the administration of the cell population and photodynamic therapy or radiotherapy are performed simultaneously or sequentially at regular intervals.

19. A method for treating a cancer patient, comprising:
administering a therapeutically effective amount of a ROCK inhibitor and an immune checkpoint inhibitor to the cancer patient.

20. The method according to claim 19, wherein the immune checkpoint inhibitor is a PD-1/PD-L1 interaction inhibitor or a CTLA-4/B7-1/B7-2 interaction inhibitor.

21. The method of claim 20, wherein the immune checkpoint inhibitor is a PD-1/PD-L1 interaction inhibitor selected from pembrolizumab, nivolumab, atezolizumab and avelumab.

22. The method of claim 20, wherein the immune checkpoint inhibitor is the CTLA-4/B7-1/B7-2 interaction inhibitor ipilimumab.

23. The method according to claim 19, further comprising administering an immunogenic cell death-inducing chemotherapeutic and/or applying photodynamic therapy or radiotherapy to the subject.

24. The method according to claim 15, wherein the photodynamic therapy is applied by administering a photosensitizer and irradiating light capable of activating the photosensitizer.
